(19) United States
(12) Patent Application Publication
(10) Pub. No.: US 2023/0070705 A1
(43) Pub. Date: Mar. 9, 2023

(54) FLUIDIC DEVICES WITH EXTRACTABLE IN-SITU-FORMED HYDROGEL STRUCTURES INTERFACED WITH FLUIDIC CHANNELS AND METHODS OF USE THEREOF

(71) Applicant: THE GOVERNING COUNCIL OF THE UNIVERSITY OF TORONTO, TORONTO (CA)

(72) Inventors: EDMOND YOUNG, TORONTO (CA); SIWAN PARK, TORONTO (CA)

(21) Appl. No.: 17/940,766

(22) Filed: Sep. 8, 2022

Related U.S. Application Data
(60) Provisional application No. 63/241,884, filed on Sep. 8, 2021.

(51) Int. Cl. C12M 3/06; C12N 5/071; C12M 1/34; C12M 1/12; C12M 1/36; C12M 1/00

(52) U.S. Cl. CPC C12M 23/16; C12M 25/02; C12M 29/26; C12M 41/44; C12M 41/48; C12N 5/0688

(57) ABSTRACT

Fluidic devices are provided and/or configured to form and support, extractable in-situ-formed hydrogels or hydrogel membranes that reside in a hydrogel chamber formed above, and in direct fluid communication with, an underlying fluidic channel, in the absence of an intervening membrane. In some example embodiments, the integrated fluidic device may include a geometrical hydrogel retention structure that provides a restoring force to the hydrogel when fluidic pressure is applied to the hydrogel from the underlying fluidic channel, or a geometrical meniscus-pinning feature that resists flow of a hydrogel precursor solution out of the hydrogel chamber, facilitating the formation of a hydrogel membrane extending over the integrated fluidic channel. The hydrogel or hydrogel membrane may be seeded with cells by delivering a cell-containing liquid to the fluidic channel, optionally while contacting the hydrogel with media provided in a media reservoir residing above the hydrogel layer.

### CROSS REFERENCE TO RELATED APPLICATION

This application claims priority to U.S. Provisional Pat. Application No. 63/241,884, titled “EXTRACTABLE FLOATING LIQUID GEL-BASED ORGAN-ON-A-CHIP” and filed on Sep. 8, 2021, the entire contents of which is incorporated herein by reference.

### BACKGROUND

The present disclosure relates to tissue engineering and organ-on-a-chip devices.

The COVID-19 pandemic has raised immense public awareness of respiratory health around the world. Yet, even before COVID-19 emerged in early 2020, chronic lung diseases (CLDs) such as chronic obstructive pulmonary disease (COPD), bronchitis, and asthma were already becoming a major global health challenge responsible for millions of deaths globally each year.[1] Every time we breathe, our lungs are exposed to airborne pathogens (including viruses), environmental toxicants, and indoor and outdoor pollutants that either cause respiratory diseases or exacerbate underlying illnesses.[2-4] New drugs for CLDs are urgently needed, but the development of new therapeutics for CLDs is slow and costly because of high failure rates.[5] The successful development and manufacturing of COVID-19 vaccines have been the exception in the drug development space, owing to the massive collective efforts of the international research community and the urgent need to limit the dangerous spread and lethal impact of COVID-19. Aside from this example, however, high attrition rates during the drug development process are the norm. A major problem in drug development that contributes to high failure rates is that existing experimental models for testing drug delivery and pharmacological activity do not properly represent how real human tissues interact with drugs. In respiratory research specifically, experimental models must overcome two main challenges: (i) accurately modelling the complex microenvironment of lung tissues, and (ii) accurately modelling the delivery and exposure of airborne substances to the tissue.

The respiratory epithelium serves as the first line of defense against environmental agents that enter the respiratory tract. The epithelium lines the inner wall of lung airways and the inner surface of alveoli. To understand the interactions between environmental agents and the epithelial surface, the mechanisms of lung disease, and the effects of drugs on respiratory health, it is necessary to examine how epithelial cells behave, function, and adapt to different insults or stimuli, ideally within a tissue microenvironment that mimics native airways.

Airborne substances enter the lung via inhalation and are carried by airflow into the various branches of the respiratory tree. Airways in different generations of the respiratory tree experience different airflow rates, which impose different shear stresses on the epithelium leading to mechanobiological stimulation of the epithelium. Particulate matter and other airborne substances carried by airflow then deposit on the epithelium via impaction, gravitational sedimentation, and Brownian diffusion depending on the locations of the airway and the characteristics of the particulates.[6] Most of the deposited particles are removed by mucociliary clearance, a transport mechanism involving the coordinated movement of beating cilia on the apical surface of the epithelium and the mucus layer resting above the cilia. However, particle deposition and retention within the airway are much more complex phenomena that involve other factors besides particle size and number. Other factors that have contributing roles include the lung airway surface, dynamics of particles caused by chemical composition or aggregation, and the shape and surface chemistry of the particles.[7] Dysfunction of mucociliary clearance is commonly associated with various CLDs,[8] with various factors including ciliary beating frequency, mucus secretion rates, and mucin composition contributing to the regulation of mucociliary clearance. [9,10] Since goblet cells and ciliated cells within the epithelium are responsible for producing mucin and generating movement of cilia, respectively, the ability to recapitulate accurate proportions of different cell populations within the epithelium is critical to creating and maintaining effective in vitro models of airway epithelium. Importantly, the quality of airflow on the epithelium is vital to the airway microenvironment as it governs both mucociliary transport as well as epithelial barrier function.[11,12] Yet, despite the importance of airflow on the biological relevance of epithelial tissue models, many researchers neglect to apply physiological airflow on epithelium and do not examine the potential impact of airflow absence on the physiological relevance of their in vitro models.

One of the most popular and common in vitro formats for airway epithelial cells is culturing on Transwell membrane inserts and creating an air-liquid interface (ALl) above the epithelial cells to induce apicobasal polarization.[13] Successful ALI culture results in expression of tight junctions, motile cilia, and viscous mucus, which are all indicative of the morphology of airway epithelium.[14][15] However, mucociliary differentiation by ALI has been shown to require approximately three weeks of culture and maintenance; such long-term culture increases the potential for contamination, dehydration, and lower cell viability.[16] In addition, Transwell membrane inserts are 2D polymeric substrates that are not easily amenable to airflow and are also biologically inert, lacking the proper 3D extracellular matrix (ECM) components that comprise the lamina propria of airway tissues. Elad et al. developed a parallel plate flow chamber system that enables airflow exposure over epithelium cultured in a Transwell insert,[17,18] but such large parallel plate flow systems are not scalable and are therefore not suitable for drug screening applications. Moreover, the issue of bioinertness of the membrane still remains even though airflow can be achieved.

The recent emergence of microfluidic “organ-on-a-chip” (OOC) systems have enabled the recapitulation of critical spatiotemporal features of complex tissue microenvironments in vitro. [19-21] Recapitulating lung environment on a microfluidic device was first introduced by Huh et al., who showed a successful fabrication of PDMS-based membrane device to mimic the blood-air barrier of an alveolus.[22] Its main feature is the cyclic stretching of its PDMS membrane to mimic the breathing motion of the lung and stretching of the alveolus; however, the elastomeric membrane does lack the physicochemical properties and bioactivity of the matrix found in the native basement membrane. Recently, Zamprogno et al. developed a “second-generation” lung-on-a-chip using a biodegradable collagen and elastin membrane on a hexagonal golden mesh structure.[23] Although the biocompatibility of this mesh-supported membrane overcomes some constraints of PDMS-based membranes, downstream analyses in this system remain limited to on-chip assessments.

The present inventors previously developed an airway-on-a-chip with a matrix-based hydrogel that could accommodate the coculture of airway epithelial and bronchial smooth muscle cells while also mimicking the lamina propria layer between the two cell types in bronchioles,[24] but this previous design was only tested under static conditions, and did not include any design elements to anchor the hydrogel and prevent gel detachment or leakage during sustained airflow. Note that while a number of lung-on-a-chip devices focus on cyclic stretching as the dominant mechanical stimulus,[24] not many focus on the effect of airflow-induced shear stress. Thus, there is a critical need to develop an in vitro platform for airway studies that has the potential for increased throughput, allows mechano-stimulation via airflow, enables cell-matrix interactions, and is amenable to both on-chip and off-chip downstream analyses for more advanced readouts.

### SUMMARY

Fluidic devices are provided comprising, and/or configured to form and support, extractable in-situ-formed hydrogels or hydrogel membranes that reside in a hydrogel chamber formed above, and in direct fluid communication with, an underlying fluidic channel, in the absence of an intervening membrane. In some example embodiments, the integrated fluidic device may include a geometrical hydrogel retention structure that provides a restoring force to the hydrogel when fluidic pressure is applied to the hydrogel from the underlying fluidic channel, or a geometrical meniscus-pinning feature that resists flow of a hydrogel precursor solution out of the hydrogel chamber, facilitating the formation of a hydrogel membrane extending over the integrated fluidic channel. The hydrogel or hydrogel membrane may be seeded with cells by delivering a cell-containing liquid to the fluidic channel, optionally while contacting the hydrogel with media provided in a media reservoir residing above the hydrogel layer.

Accordingly, in a first aspect, there is provided a fluidic device comprising:
a multilayer fluidic structure having formed therein:
   a fluidic channel;
   a hydrogel chamber residing above the fluidic channel, the hydrogel chamber being defined at least in part by a side wall and a base surface, the base surface having an aperture defined therein such that the hydrogel chamber is in direct fluid communication with the fluidic channel through the aperture, and such that when a hydrogel is formed within the hydrogel chamber with the hydrogel contacting the base surface and extending across the aperture, the fluidic channel is in direct fluidic communication with a lower surface of the hydrogel in absence of an intervening membrane, the lower surface of the hydrogel being exposed to the fluidic channel through the aperture; and
   a media reservoir residing above the hydrogel chamber, the media reservoir being in fluid communication with the hydrogel chamber, such that when the hydrogel is formed within the hydrogel chamber and liquid media is provided to the media reservoir, the liquid media is in fluid contact with an upper surface of the hydrogel;
the hydrogel chamber comprising a geometrical hydrogel retention structure configured to provide a restoring force to the hydrogel when fluidic pressure is applied to the lower surface of the hydrogel from the fluidic channel.

In some example implementations of the device, the hydrogel chamber and the aperture are configured such that when a hydrogel precursor solution is dispensed such that the hydrogel precursor solution contacts the base surface, the hydrogel precursor solution extends across the aperture without flowing into the fluidic channel, thereby facilitating in-situ formation of the hydrogel within the hydrogel chamber.

In some example implementations of the device, the geometrical hydrogel retention structure comprises a hydrogel retention lip extending from the side wall at a location remote from the base surface, such that when the hydrogel is formed within the hydrogel chamber with an upper surface of the hydrogel contacting a lower surface of the hydrogel retention lip, the hydrogel retention lip provides, at least in part, the restoring force to the hydrogel when fluidic pressure is applied to the lower surface of the hydrogel from the fluidic channel.

In some example implementations of the device, the geometrical hydrogel retention structure comprises one or more protrusions extending from the base surface, such that when the hydrogel is formed within the hydrogel chamber with the hydrogel at least partially surrounding the protrusions, the protrusions provide, at least in part, the restoring force to the hydrogel when fluidic pressure is applied to the lower surface of the hydrogel from the fluidic channel.

In some example implementations of the device, at least one protrusion is a micropost.

In some example implementations of the device, at least a portion of the base surface extends across the fluidic channel, thereby forming a lower lip feature configured to resist flow of a hydrogel precursor solution into the fluidic channel when the hydrogel precursor solution is dispensed into the hydrogel chamber.

In some example implementations, the device includes the hydrogel within the hydrogel chamber.

In some example implementations of the device, the fluidic channel, the hydrogel chamber and the media reservoir define a first fluidic network, the multilayer fluidic structure comprising at least one additional fluidic network.

In another aspect, there is provided a method of forming a hydrogel in-situ within a fluidic device, the method comprising:
providing the fluidic device as described above;
dispensing a hydrogel precursor solution such that the hydrogel precursor solution contacts the base surface and the geometrical hydrogel retention structure, and such that the hydrogel precursor solution extends across the aperture without flowing into the fluidic channel; and
hardening the hydrogel precursor solution to form the hydrogel in-situ within the hydrogel chamber, such that the hydrogel contacts, at least in part, the geometrical hydrogel retention structure.

In some example implementations, the method further includes providing a cell-containing liquid to the fluidic channel and incubating the fluidic device to facilitate adhesion of cells of the cell-containing liquid to the lower surface of the hydrogel exposed by the aperture.

In some example implementations, the method includes providing liquid media to the media reservoir and incubating the fluidic device.

In some example implementations, the method further includes removing the cell-containing liquid from the fluidic channel; and delivering a fluid to the fluidic channel to expose the cells formed on the lower surface of the hydrogel to the fluid.

In some example implementations of the method, the fluid comprises a gas, and wherein the hydrogel is secured by the geometrical hydrogel retention structure such that a seal is maintained between the hydrogel and the fluidic channel during delivery of the gas.

In some example implementations of the method, the cells are airway epithelial cells, wherein the fluid comprises air, and wherein the air is delivered at a flow rate mimicking a physiological flow rate.

In some example implementations of the method, the fluid comprises particulate matter.

In some example implementations, the method further includes extracting the hydrogel from the hydrogel chamber, thereby obtaining an extracted hydrogel; and performing one or more analytical procedures to characterize the cells of the extracted hydrogel.

In another aspect, there is provided a fluidic system comprising:
a fluidic device as described above;
a fluid source;
a fluid delivery apparatus in fluid communication with the fluid source and an inlet of the fluidic channel; and
control circuitry operatively coupled to the fluid delivery apparatus, the control circuitry being configured to control the fluid delivery apparatus to deliver a fluid from the fluid source to the fluidic device.

In some example implementations, the fluidic system further includes a mixer in fluid communication with a particulate matter source and a fluidic path extending from the fluid delivery apparatus to the fluidic device, the mixer being configured for injection of particulate matter into the fluid delivered to the fluidic device.

In another aspect, there is provided a fluidic device comprising:
a multilayer fluidic structure having formed therein:
a fluidic channel;
a hydrogel chamber residing above the fluidic channel, the hydrogel chamber being defined at least in part by a side wall and a base surface, the base surface having an aperture defined therein such that the hydrogel chamber is in direct fluid communication with the fluidic channel through the aperture;
a media reservoir residing above the hydrogel chamber, the media reservoir being in fluid communication with the hydrogel chamber; and
a geometrical meniscus-pinning feature configured such that when a hydrogel precursor solution is delivered to the hydrogel chamber for in-situ formation of a hydrogel therein, the geometrical meniscus-pinning feature resists flow of the hydrogel precursor solution out of the hydrogel chamber, thereby preventing contact of the hydrogel precursor solution with one or more surfaces of the media reservoir;
the geometrical meniscus-pinning feature thereby confining formation of the hydrogel within the hydrogel chamber, such that subsequent drying of the hydrogel results in formation a hydrogel membrane secured to the base surface and extending over the aperture, and such that the fluidic channel is in direct fluidic communication with a lower surface of the hydrogel membrane in absence of an intervening additional membrane, the lower surface of the hydrogel membrane being exposed to the fluidic channel through the aperture.

In some example implementations of the fluidic device, the geometrical meniscus-pinning feature comprises a ridge.

In some example implementations of the fluidic device, the aperture is a first aperture and the base surface is a first base surface, and wherein the media reservoir is defined in part by a second base surface having a second aperture defined therein, such that the media reservoir is in fluid communication with the hydrogel chamber through the second aperture, and wherein the geometrical meniscus-pinning feature resides on a portion of the second base surface that lies adjacent to the second aperture.

In some example implementations, the fluidic device further includes the hydrogel membrane extending over the aperture.

In some example implementations of the fluidic device, the fluidic channel, the hydrogel chamber and the media reservoir define a first fluidic network, the multilayer fluidic structure comprising at least one additional fluidic network.

In another aspect, there is provided a method of forming a hydrogel membrane in-situ within a fluidic device, the method comprising:
providing the fluidic device for forming a hydrogel membrane, as described above;
dispensing a hydrogel precursor solution such that the hydrogel precursor solution contacts the base surface, and such that the hydrogel precursor solution extends across the aperture without flowing into the fluidic channel, and such that the geometrical meniscus-pinning feature prevents the hydrogel precursor solution from flowing into the media reservoir;
hardening the hydrogel precursor solution to form the hydrogel in-situ within the hydrogel chamber; and
drying, at least in part, the hydrogel to form the hydrogel membrane, wherein the geometrical meniscus-pinning feature facilitates shrinkage of the hydrogel within the hydrogel chamber to form the hydrogel membrane such that the hydrogel membrane is secured to the base surface and extends over the aperture.

In some example implementations, the method further includes providing a cell-containing liquid to the fluidic channel and incubating the fluidic device to facilitate adhesion of cells of the cell-containing liquid to the lower surface of the hydrogel membrane exposed by the aperture.

In some example implementations, the method further includes providing liquid media to the media reservoir and incubating the fluidic device.

In some example implementations, the method further includes removing the cell-containing liquid from the fluidic channel; and delivering a fluid to the fluidic channel to expose the cells formed on the lower surface of the hydrogel membrane to the fluid.

In some example implementations of the method, the fluid comprises a gas, and wherein the hydrogel membrane is secured by to the base surface of the hydrogel chamber such that a seal is maintained between the hydrogel membrane and the fluidic channel during delivery of the gas.

In some example implementations of the method, the cells are airway epithelial cells, wherein the fluid comprises air, and wherein the air is delivered at a flow rate mimicking a physiological flow rate.

In some example implementations of the method, the fluid comprises particulate matter.

In some example implementations, the method further includes extracting the hydrogel membrane from the hydrogel chamber, thereby obtaining an extracted hydrogel membrane; and performing one or more analytical procedures to characterize the cells of the extracted hydrogel.

In another aspect, there is provided a fluidic system comprising:
a fluidic device for forming a hydrogel membrane, as described above;
a fluid source;
a fluid delivery apparatus in fluid communication with the fluid source and an inlet of the fluidic channel; and
control circuitry operatively coupled to the fluid delivery apparatus, the control circuitry being configured to control the fluid delivery apparatus to deliver a fluid from the fluid source to the fluidic device.

In some example implementations, the fluidic system further includes a mixer in fluid communication with a particulate matter source and a fluidic path extending from the fluid delivery apparatus to the fluidic device, the mixer being configured for injection of particulate matter into the fluid delivered to the fluidic device.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIGS. 1A, 1B and 1C illustrate three different example integrated hydrogel-supporting fluidic devices, with FIG. 1A showing an open-top device, FIG. 1B showing an example device with a top cover enclosing the central portion of the media reservoir and the hydrogel chamber, and FIG. 1C showing an example of an open-top integrated hydrogel-supporting fluidic device that is absent of microposts. By extension, an embodiment that is absent of microposts can also include a top cover enclosing the central portion of the media reservoir and the hydrogel chamber.

FIG. 2 is a cross-section of an example device illustrating use of the device for investigating the response of epithelial airway cells, formed on the exposed lower surface of the in-situ-grown hydrogel, to applied airflow and exposure to particulate matter.

FIGS. 3A, 3B and 3C illustrate various example integrated hydrogel-supporting fluidic device that are configured to support and secure an in-situ-grown hydrogel membrane over an underlying fluidic channel.

FIGS. 4A, 4B, 4C, 4D, 4E and 4F illustrate various example device configurations, with a detailed isometric view showing a cutaway of the hydrogel chamber, including: an example configuration with an open top (FIG. 4A), an example configuration with a closed ceiling, (FIG. 4B), an example open-top configuration without microposts (FIG. 4C), an example configuration for forming a thin hydrogel membrane (FIG. 4D), an alternative example configuration for forming a thin hydrogel membrane involving branched membranes (FIG. 4E), and an example configuration for forming two thin hydrogel membranes in parallel independent pockets and involving exposure of each membrane to their own underlying fluidic channel (FIG. 4F).

FIG. 5 is an example of a system for controlling fluid delivery to an integrated hydrogel-supporting fluidic device.

FIGS. 6A, 6B, 6C, 6D, 6E, 6F and 6G illustrate an integrated hydrogel-supporting fluidic device and airflow system. (a) Photograph of fabricated example plastic integrated hydrogel-supporting fluidic device consisting of an array of 4 integrated hydrogel-supporting fluidic culture systems. (b) Exploded view of individual layers of the integrated hydrogel-supporting fluidic device. From top layer to bottom: Port layer (with media reservoir), gel-containment lip layer, microanchored gel layer, and airflow microchannel layer. (c) Magnified image of milled microanchors that secure the suspended hydrogel. (d) Extracted hydrogel to be prepared for downstream analyses. (e) Isometric and cross-sectional views of the integrated hydrogel-supporting fluidic device. (f) Cross-section of the device showing location of gel, epithelium cultured on underside of gel, and airborne particles delivered via the airflow system. (g) Schematic of airflow system setup consisting of gas cylinder for air source, air filter, mass flow controller for flow rate control, bubbler humidifier for humidity control, and hygrometer and thermometer for monitoring air quality. Airflow tube was heated to provide body temperature air on the epithelium. Scalebars: (a) 1 cm, (c) 1.5 mm, and (d) 1 mm.

FIGS. 7A, 7B, 7C, 7D and 7E show modelling flow in the integrated hydrogel-supporting fluidic device and matching airway flow rates. (a) Isometric drawing of integrated hydrogel-supporting fluidic system showing the three main fluid domains: the airflow microchannel, the suspended hydrogel, and the upper media reservoir. (b) COMSOL simulation showing straight streamlines of flow and simulated parabolic velocity profile in the lower microchannel. (c) Wall shear stress by airway generation, based on study by Weibel.[32-33] (d-e) Shear stress magnitude on the microchannel ceiling (hydrogel underside) showing uniform shear on entire gel surface (dots = COMSOL; dotted line = theoretical Purday approximation).

FIGS. 8A, 8B, 8C and 8D illustrate the effects on airway epithelium morphology and cell composition under three different culture conditions: (i) submerged (in media) for 96 h, (ii) static air-liquid interface (ALI) for 96 h, and (iii) static ALI for 48 h plus airflow for 48 h. (a) Schematic of experimental culture procedure, including 10 days of submerged static culture to reach confluence in all cases prior to testing the 3 different conditions. (b) Immunostaining images for all 3 culture conditions. Left column: ZO-1, MUC5AC and Hoechst stain for nuclei. Right column: acetylated a-tubulin and Hoechst. Scalebar = 50 µm. (c) Number of goblet cells per 100 total cells for each culture condition. (* p < 0.05, n = 3). (e) ZO-1 expression measured as % area coverage of positive ZO-1 stain.

FIGS. 9A, 9B, 9C, 9D, 9E and 9F are scanning electron microscopy (SEM) images of airway epithelial cells (AECs) under different culture conditions. (a) AECs in submerged culture for 96 h post-confluence. (b) AECs in static ALI culture for 96 h post-confluence. (c) AECs in static ALI culture for 30 days in a Transwell insert. (d) AECs cultured with airflow at 80% relative humidity for 24 h post-confluence. (e-f) AECs in static ALI culture for 48 h followed by airflow at 95% relative humidity for (e) 24 h and (f) 48 h. Scalebars = 1 mm.

FIGS. 10A-10G show histology sectioning and hematoxylin and eosin staining of extracted floating gels with AECs. (a) Illustration showing the direction of the sectioning. (b) Calu-3 cells submerge-cultured for 96 h post-confluence. (c) Calu-3 cells cultured under static ALI condition for 96 h post-confluence. (d-e) AECs in static ALI culture for 48 h followed by airflow for 48 h post-confluence (two separate trials). Note the morphological difference between two samples of AECs under the same airflow and culture conditions. (f) AECs submerge-cultured on the transwell membrane for 37 days. (g) AECs cultured under static ALI for 30 days post-confluence (7 days to reach confluency). Black arrows indicate the ciliated regions on the apical side of the epithelia. Scalebars = 50 mm.

FIGS. 11A, 11B, 11C, 11D and 11E show particle deposition on airway epithelium cultured on the floating gel. (a) Schematic of delivery. Particles were delivered as a bolus injection from the syringe, mixed into the airflow, and deposited on the epithelium. (b-d) Carbon black deposited on the Calu-3 epithelial cell. Scalebar: (b) 100 mm, (c)10 mm (d) 1 mm. (e) EDAX scan with silicon drift detector on the SEM sample to confirm the carbon content in the deposited material. Graph represents one-dimensional map of carbon (white) and oxygen (black) in the scanned area.

FIG. 12 shows measurements of relative humidity over 48 hours of airflow application.

FIGS. 13A, 13B and 13C are images showing morphology of AECs cultured on the hydrogel versus PMMA surface. (a) Selected region within the bottom channel of the integrated hydrogel-supporting fluidic device where cells cultured in both surfaces were visible. (b) AECs cultured on the surface of the hydrogel. (c) AECs cultured on the surface of the PMMA.

FIG. 14 provides a schematic illustration and photographs of an example implementation of an extractable floating liquid-gel-based organ-on-a-chip system.

FIGS. 15A, 15B, 15C and 15D illustrate a fabrication methodology and preliminary characterization of collagen membrane devices. (A) The collagen membrane is fabricated in situ by first seeding a collagen hydrogel in the open-bottom well, situated directly above the lower channel, indicated in the left image and then allowing it to dry, producing a thin collagen film suspended in place. The hydrogel is maintained over the well opening by surface tension forces during polymerization. The membrane fabrication geometry can be bonded to any lower microchannel geometry prior to membrane seeding, an example of which is shown in light grey, thus enabling fluidic access to both the top surface through the open well and the bottom surface through the underlying channels. (B) Example optical cross-sections of collagen membranes, and the measured thickness variation as a function of the product of the initial hydrogel thickness and collagen concentration. (C, D) An example of a tissue model constructed using the device shown in A, consisting of adjacent monolayers of Calu-3 and human umbilical vein endothelial cells (HUVECs) separated by a collagen membrane.

FIGS. 16A, 16B, 16C, 16D, 16E, 16F, 16G and 16H show morphologies of airway epithelial cells cultured under different conditions in the membrane-integrated E-FLOAT. (A, C, E) Images of submerge-cultured Calu-3 cells showing expressed ciliary structures and tight junctions. Notice that in C and E, which are cross-sectional images of Calu-3 cells, that the cells are cuboidal and not polarized, as ciliary structures (yellow = α-acetylated tubulin) are expressed around the cell membrane. Green = ZO-1 (tight junctions). (B, D, F) Images of the Calu-3 cells cultured under the air-liquid interface condition for 120 hours. Cross-sectional images show the ciliary structure that is expressed on the apical side of the cells as cells are polarized and pseudostratified. (G) Mucus-producing goblet cell differentiations (red = MUC5AC) under the airflow condition for 48 hours followed by 72 hours of ALI culture. (H) Mucus-producing goblet cell differentiations for submerge-cultured Calu-3 cells.

FIG. 17A provides a schematics of a bidirectional airflow system. The syringe pump is infusing and withdrawing the warm and humidified air to provide bidirectional airflow. The mass flow controller is humidifying the air contained in the humidifier.

FIG. 17B is a graph that shows the measured pressure near the outlet port of the device when 20ml/min of air is infused/withdrew from the syringe pump.

FIG. 17C is a flow rate conversion chart. Lung has 23 generations of airways and each experience different flow rates. Vavr@chip(m/s) column shows the converted values of flow rate required in the device channel. The Estimated VFR column shows the volumetric flow rate that needs to be provided by the syringe pump in ml/min scale.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

According to various example embodiments of the present disclosure, fluidic devices are provided comprising, and/or configured to form and support, extractable in-situ-formed hydrogels or hydrogel membranes that reside in a hydrogel chamber formed above, and in direct fluid communication with, an underlying fluidic channel, in the absence of an intervening membrane. Such a device is henceforth referred to as an “integrated hydrogel-supporting fluidic device”.

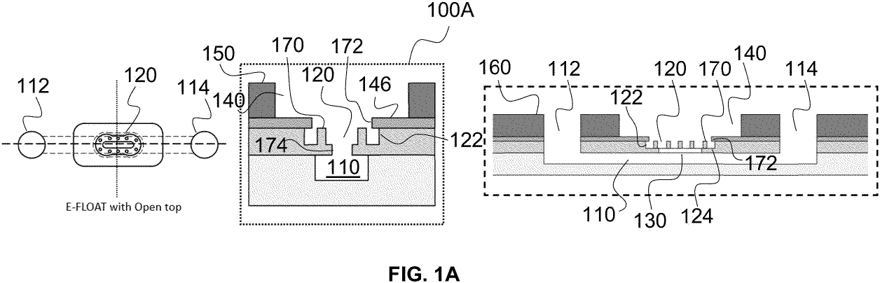

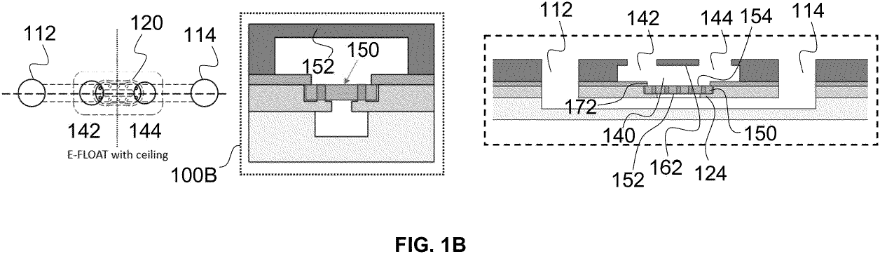

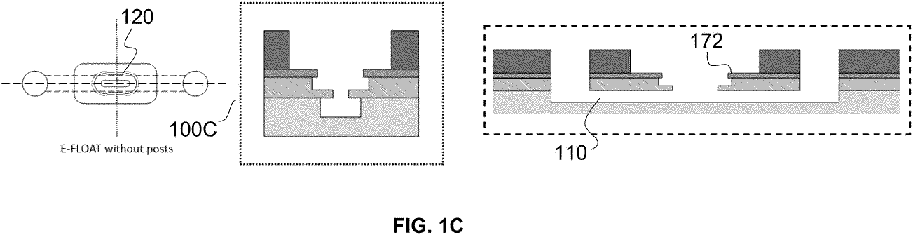

FIG. 1A illustrates an example embodiment of an integrated hydrogel-supporting fluidic device. The device includes a multilayer fluidic structure 100A, which is shown from overhead (left) and orthogonal vertical cross-sections (middle and right). A fluidic channel 110, which may be a microfluidic channel, extends beneath a hydrogel chamber 120 within a lower layer of the device, and is externally addressable via ports 112 and 114.

The hydrogel chamber 120, which resides above the fluidic channel 110, is bounded by a side wall 122 and a base surface 124. The base surface 124 includes an aperture 130 that brings the hydrogel chamber 120 in direct fluid communication with the fluidic channel 110. A media reservoir 140 is located above the hydrogel chamber 120. As will be described in further detail below, the base surface 124 of the hydrogel chamber and the aperture 130 are provided with dimensions such that when a hydrogel precursor solution is dispensed into the hydrogel chamber 120 and contacts the base surface 124, the hydrogel precursor solution extends across the aperture 130 without flowing into the underlying fluidic channel 110 due to surface tension forces, thereby facilitating in-situ formation of the hydrogel in the hydrogel chamber 120.

In this embodiment illustrated in FIG. 1A, the top chamber serves as a static reservoir of culture media. While FIG. 1A illustrates an example implementation in which the media reservoir 140 and the hydrogel chamber 120 are open, FIG. 1B illustrates an alternative example implementation in which an upper device layer 160 covers at least a portion of the media reservoir 140 and the hydrogel chamber 120.

As can be seen in FIG. 1B, the central portion of the media reservoir 140 is covered by the central portion 162 of the upper device layer 160, with additional ports 142 and 144 providing external access to the media reservoir 140 and the hydrogel chamber 120. The topmost device layer includes a ceiling that encloses the top chamber or channel. This topmost ceiling includes inlet and outlet access ports/holes 142 and 144, but otherwise maintains fluid contained within the top chamber and also enables interconnects and attachment of tubing to the inlet and outlet ports to facilitate perfusion flow as needed.

While FIG. 1A illustrates an example case in which the fluidic device 100A is empty and has not yet been loaded with a hydrogel, FIG. 1B illustrates an example case in which a hydrogel 150 has been formed in-situ within the fluidic device 100B. The hydrogel 150 has been formed in-situ by dispensing a hydrogel precursor solution into the hydrogel chamber 120 and hardening the hydrogel precursor solution to form the hydrogel 150. As can be seen in the figure, the hydrogel 150 contacts the base surface 124 and extends across the aperture 130, such that the fluidic channel 110 is in direct fluidic communication with a lower surface 152 of the hydrogel 150, in absence of an intervening membrane. The lower surface 152 of the hydrogel 150 is thus exposed to the fluidic channel 110 through the aperture 130.

FIG. 1B also illustrates how the media reservoir 140 is in fluid communication with the hydrogel 150 within the hydrogel chamber 120, such that when liquid media is provided to the media reservoir 140, the liquid media is in fluid contact with an upper surface 154 of the hydrogel 150.

Unlike previously known fluidic devices that interface a hydrogel structure with a fluidic channel within a multilayer fluidic device, each of the example embodiments illustrated in FIGS. 1A, 1B and 1C includes at least one geometrical hydrogel retention structure that provides a restoring force to a hydrogel 150 formed within the hydrogel chamber 120 when fluidic pressure is applied to the hydrogel 150 from the underlying fluidic channel 110. FIGS. 1A and 1B illustrate example embodiments that include two different types of geometrical hydrogel retention structures.

A first example of a geometrical hydrogel retention structure illustrated in FIGS. 1A and 1B are the protrusions 170 extending from the base surface 124 of the hydrogel chamber 120. The protrusions, which are embedded, at least in part, within the hydrogel 150 that is formed in-situ within the hydrogel chamber 120, provide a restoring (e.g. anchoring) force (e.g. a shear force) that enables the hydrogel to withstand a higher fluidic pressure applied within the underlying fluidic channel 110 while remaining attached within the hydrogel chamber 120. For example, the protrusions may enable a leak-free seal to persist between the hydrogel 150 and the underlying fluidic channel 110 beyond a threshold pressure that would result in the absence of the protrusions. While the figures illustrate the example case of the protrusions 170 being provided in the form of micropillars (or micro posts or microanchors), it will be understood that a wide variety of geometrical shapes may be employed to form the protrusions. Moreover, protrusions can be any shape, such as, but not limited to, triangular, semicircular, square, and c-shaped.

FIGS. 1A and 1B also illustrate an additional type of a geometrical hydrogel retention structure that is provided in the form of a hydrogel retention lip 172. The hydrogel retention lip 172 extends from the side wall 122 of the hydrogel chamber 120 at a location remote from the base surface 122, for example, at the top of the side wall 122. As shown in FIG. 1A, the hydrogel retention lip 172 may be provided as an extension of a base surface 146 of the media reservoir, Accordingly, when the hydrogel 150 is formed within the hydrogel chamber 120 with an upper surface of the hydrogel 154 contacting a lower surface of the hydrogel retention lip 172, the hydrogel retention lip provides 172, at least in part, a restoring force to the hydrogel 150 when fluidic pressure is applied to the lower surface 152 of the hydrogel 150 from the fluidic channel 110.

In the example configuration shown in FIG. 1B, the hydrogel retention lip 172 secures the suspended hydrogel from a vertical shift in position, while the microposts 170 further anchor the suspended hydrogel in its lateral position, preventing shrinkage and detachment from side walls 122 in the case where contractile cells may be embedded in the hydrogel leading to gel contraction.

In the example embodiments illustrated in FIGS. 1A and 1B, at least a portion of the base surface 124 of the hydrogel chamber 120 extends above the fluidic channel 110, thereby forming a lower lip feature 174 configured to resist flow of the hydrogel precursor solution into the fluidic channel 110 when the hydrogel precursor solution is dispensed into the hydrogel chamber 120.

FIG. 1C illustrates an alternative example integrated hydrogel-supporting fluidic device 100C that includes the hydrogel retention lip 172 but is absent of protrusions extending from the base surface 124. It has been found by the inventors that in some cases, such a configuration may be sufficient to secure and stabilize the hydrogel in the presence of pressure applied via the fluidic channel 110. In such a case, the suspended hydrogel may support epithelial monolayer cultures, but may not support embedded culture of contractile cells in the hydrogel which may lead to gel contraction.

The example multilayer devices shown in FIGS. 1A-1C illustrate several configurations in which components of the multilayer integrated hydrogel-supporting fluidic devices are incorporated into specific layers. It will be understood, however, that these examples configurations are not intended to be limiting and that other device variations are possible and contemplated by the present disclosure. For example, while the devices are shown as four-layer devices, the number of device layers may differ from four. In another example, while the hydrogel retention lip 172 is shown in FIG. 1A as being formed within the second layer from the top of the device, this feature may alternatively be formed within the top layer of the device. Those skilled in the art of microfluidics and integrated multilayer fluidic devices will understand that many such variations are possible without departing from the intended scope of the present disclosure.

Moreover, other example device configurations may employ alternative combinations of the features shown in FIGS. 1A, 1B and 1C. For example, in another example implementation (not shown), an integrated hydrogel-supporting fluidic device may include a lower lip feature, an upper lip feature, and be absent of both protrusions and a ceiling that encloses the top chamber.

Furthermore, although the preceding examples illustrate devices with a single fluidic network that includes the fluidic channel, hydrogel chamber and media reservoir, it will be understood that one or more of such fluidic networks may be integrated into a single multilayer integrated hydrogel-supporting fluidic device. For example, a plurality of such fluidic networks may be provided in a single device in an arrayed configuration, thereby enabling the in-situ formation and testing of multiple hydrogel constructs per device.

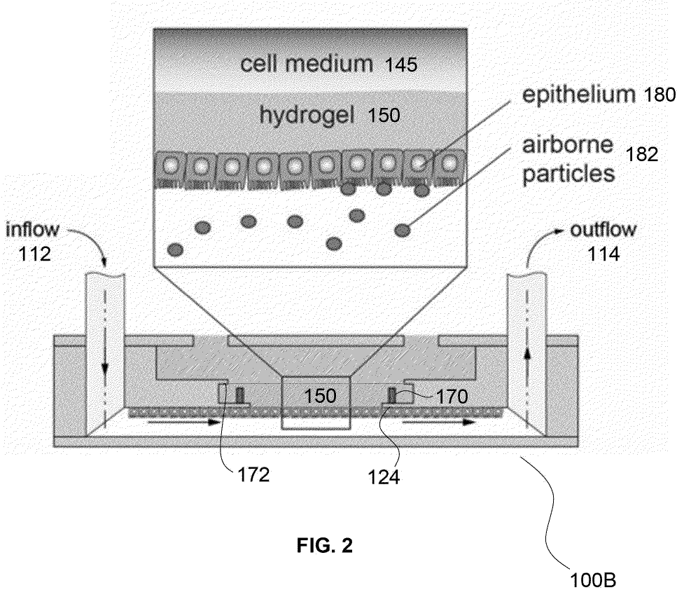

FIG. 2 illustrates an example application of the integrated hydrogel-supporting fluidic device shown in FIG. 1B. With reference to both FIG. 2 and FIG. 1B, a hydrogel precursor liquid is dispensed, through one or both of ports 142 and 144, into the hydrogel chamber 120. The hydrogel precursor solution (e.g. 7 µl per channel) may be dispensed (e.g. pipetted) along the side wall 122 of the hydrogel chamber 120, in order to guide the hydrogel precursor solution into and around the base platform of the hydrogel chamber 120. This step is beneficial, for example, in the case of a hydrogel chamber 120 that includes anchoring protrusions to ensure that the hydrogel is properly anchored when polymerized. According to one example dispensing method, once the side wall of the hydrogel chamber 120 is wetted, the central region of the hydrogel chamber may be slowly filled with the hydrogel precursor solution, such that the hydrogel precursor extends across the aperture without entering into the underlying fluidic channel.

The side walls 122 of the hydrogel chamber 120 may be configured such that the hydrogel precursor solution flows by assisted capillary action. The present inventors found that during the flow of the hydrogel precursor solution into the hydrogel chamber, external forces such as gravity were negligible. However, it was found that an excess of pipetting pressure could exceed the interfacial tension between the side wall 122 and the hydrogel precursor solution, which could lead to the hydrogel precursor passing through the aperture into the underlying fluidic channel.

When all the hydrogel chamber 120 is filled with the hydrogel precursor solution, the device may be incubated to cause hardening of the hydrogel. For example, the device may be placed in a pre-warmed humid chamber and then placed in the CO₂ incubator at 37° C. for 1 hour. After the hydrogel is polymerized, the device maybe be removed from the incubator and a cell culture solution may be dispensed into the media reservoir 140. The fluidic channel 110 may also be with the cell culture solution to rehydrate the hydrogel, and optionally to determine whether or not any leakage from the top channel to the bottom channel is present.

In some example applications, the suspended hydrogel may be employed to act as a biological scaffold to mimic the mechanical properties of the extracellular matrix (ECM). Type I collagen solution is comprised of collagen fibres. When conditions are near physiological (i.e., pH of ∼7.4 and temperature -36.5-37° C.) the fibres crosslink and form a gel-like structure. This process is similar to that of Matrigel, which consists mostly of type IV collagen and laminin. In some example implementations, the hydrogel precursor liquid may include, for example, a mixture of type I collagen and Matrigel solutions, which may be prepared in a chilled (< 4° C.) state to prevent rapid polymerization. Example final concentrations of the mixture include 6 mg/ml of Matrigel and 3 mg/ml of collagen. This example and non-limiting ratio of mixture components was tested to promote the adhesion and proliferation of Calu-3 cells in the examples described below.

A cell suspension may then be delivered to the fluidic channel 110, and the device may be subsequently incubated to facilitate cell sedimentation and attachment (optionally with the device inverted to facilitate sedimentation onto the hydrogel surface). Cell culture media may then be delivered to the fluidic chamber 110 to remove unattached cells. Cell culture media may be replenished to the media reservoir 140 and/or the fluidic channel 110 one or more times.

Having formed a cell layer along the bottom surface of the hydrogel, such as the epithelium 180, the cells may be exposed to a fluid delivered to the fluidic channel 110. It will be understood that the fluid may be a liquid or gas. In many of the non-limiting examples described below, the fluid may be air that is humidified and with which particulate matter 182 has been mixed, as illustrated in FIG. 2.

After exposing the cell layer of the hydrogel to a fluid, the hydrogel may be extracted from the integrated hydrogel-supporting fluidic device. In example implementations in which a top layer of the device encloses the hydrogel chamber, the device may be disassembled to provide access to the hydrogel. For example, a tool such as a razor blade may be employed to cleave bonded area, or the device may be configured to include two removably attachable layers, such as layers adhered via an adhesive that enables detachment. When the hydrogel chamber 200 is accessible, a tool such as a scalpel may be employed to detach the hydrogel from the hydrogel chamber. The present inventors have found that extraction of the hydrogel can be facilitated by first detaching the hydrogel from the side wall, and subsequently employing tweezers to extract the hydrogel (gel construct). If the hydrogel contains embedded cells, a stream of liquid may be pipetted under the hydrogel one or more times to slip the gel out of the hydrogel chamber. When handling the gel construct, the present inventors found that it was beneficial to minimize use of sharp tweezers to prevent any potential damage. A scooping tool may instead be employed to scoop the hydrogel from the hydrogel chamber when transferring the hydrogel to another location.

The ability to extract the hydrogel from the integrated hydrogel-supporting fluidic device expands the number of analytical methods that may be employed to characterize the hydrogel, especially given the ability of the integrated hydrogel-supporting fluidic system to be exposed to airflow and particulate matter insult. The present inventors found that the extracted hydrogels were sufficiently robust during sample handling and manipulation to permit common procedures such as immunocytochemical staining, histology sectioning, and H&E staining, as well as even more rigorous procedures such as critical point dehydration, which is necessary during sample preparation prior to SEM imaging. Based on experience handling the hydrogel samples, the extracted hydrogels were found to resemble excised tissue in tactility, and thus it is conceivable to perform other biological assays and procedures off-chip that are normally performed on ex vivo tissues, including cell lysis, cell isolation, single-cell RNA sequencing (after cell isolation), flow cytometry, matrix stiffness measurements (e.g., by atomic force microscopy), and many others. These techniques are challenging for existing lung-on-a-chip platforms with embedded polymeric membranes or meshes.

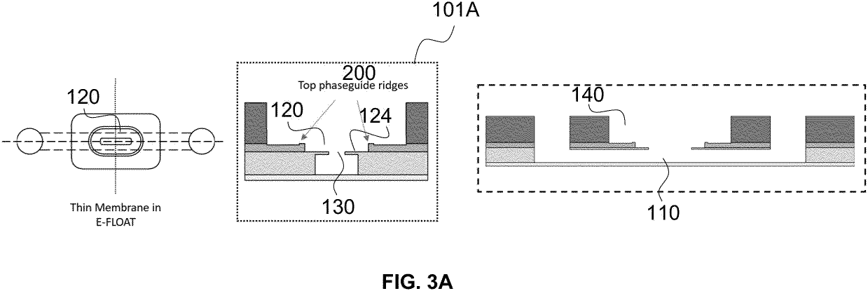

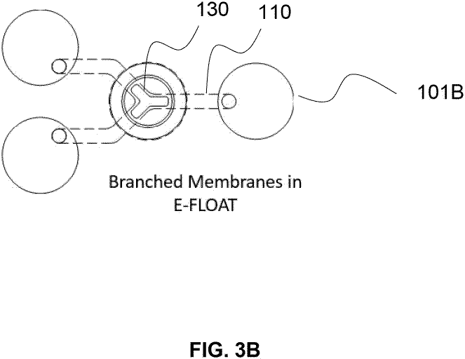

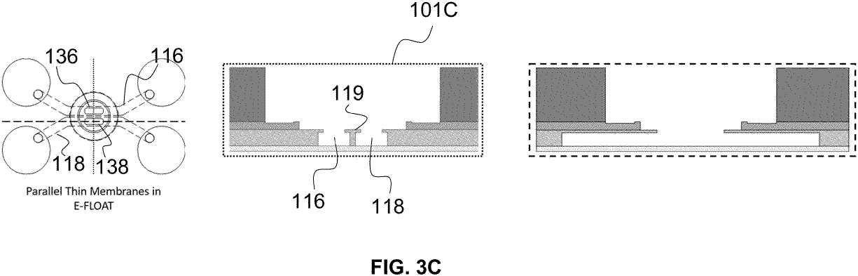

Referring now to FIGS. 3A-3C, an alternative device configuration is illustrated that facilitates the formation of a thin hydrogel membrane in-situ within an integrated hydrogel-supporting fluidic device. This device may be employed to fabricate a thin hydrogel membrane in situ by first forming a hydrogel in the hydrogel chamber 120, as per the previously described methods, and then allowing the hydrogel to dry. Drying the hydrogel results in the formation of a thin hydrogel film suspended over the aperture 130 and the underlying fluidic well 110.

In some example implementations, chemical treatment of the surface of the hydrogel chamber may be performed to facilitate or improve adhesion of the hydrogel membrane to the supporting device. For example, the hydrogel chamber 120 may be sequentially treated with sodium hydroxide and polyethylenimine (PEI) to aminate the plastic surface. The aminated surface may then be treated with glutaraldehyde, which acts as a double-headed linker molecule which covalently bonds with both the PEI and proteins in the subsequently seeded hydrogel membrane.

As shown in FIG. 3A, the example device 101A includes a geometrical meniscus-pinning feature 200 (e.g. a “phaseguide”) that is positioned such that when a hydrogel precursor solution is delivered to the hydrogel chamber 120 and substantially fills the hydrogel chamber 120, the geometrical meniscus-pinning feature 200 resists flow of the hydrogel precursor solution out of the hydrogel chamber. This facilitates the formation of a hydrogel membrane extending over the integrated fluidic channel as the hydrogel shrinks during drying becomes pinned to the base surface 124 of the hydrogel chamber 120. The hydrogel or hydrogel membrane may be seeded with cells by delivering a cell-containing liquid to the fluidic channel, optionally while contacting the hydrogel with media provided in a media reservoir residing above the hydrogel layer.

As shown in FIG. 3A, the in-situ hydrogel membrane forming device may include the media reservoir 140 disposed above the hydrogel chamber 120. This enables the dispensing of media indirectly into the media chamber 140, such that the dispensed media gently flows into the hydrogel chamber 120 to contact the hydrogel membrane, as opposed to being dispensed directly onto the hydrogel membrane, which could otherwise result in damage.

The present inventors have found that in order for the hydrogel membrane thickness to be reproducible and uniform for the example hydrogel precursor solutions used in the examples provided below, the height to width aspect ratio of the hydrogel precursor liquid should be sufficiently below one, which can be achieved by employing a hydrogel chamber 120 with a height-to-width aspect ratio of less than one. The present inventors have found that small aspect ratio ensures that the hydrogel matrix preferentially collapses downwards in a pseudo one-dimensional manner when it dehydrates, as the effect of wall adhesion is sufficiently far away from the suspended portion of the hydrogel. A larger aspect ratio results in the matrix being pulled both downwards and towards the wall as it dehydrates, and this results in inconsistent dehydration behaviour. The specific numerical aspect ratio that below which reliable membrane formation occurs is case-dependent and depends on the absolute dimensions of the membrane well and the portion of the well bottom that is open (i.e. the suspended portion of the hydrogel membrane). The skilled artisan can perform experiments to determine a suitable height-to-width aspect ratio for a given choice of hydrogel precursor liquid. In some example implementations, the aspect ratio can be less than 1, less than 0.75, or less than 0.5.

FIGS. 3B and 3C illustrate two alternative example device configurations for the formation of a hydrogel membrane based on the drying of a hydrogel that was initially formed in-situ within the hydrogel chamber. FIG. 3B illustrates an example device configuration in which the underlying fluidic channel 110 is bifurcated into two fluidic channels beneath the aperture 130, with the aperture having a Y-shape. FIG. 3C illustrates an example embodiment in which two fluidic channels 116 and 118 reside below the hydrogel chamber, separated by a channel side wall 119, with the hydrogel chamber including two per-channel apertures 136 and 138. It will be understood that the preceding embodiments configured for the in-situ formation of a hydrogel may also be adapted to include such multi-channel configurations.

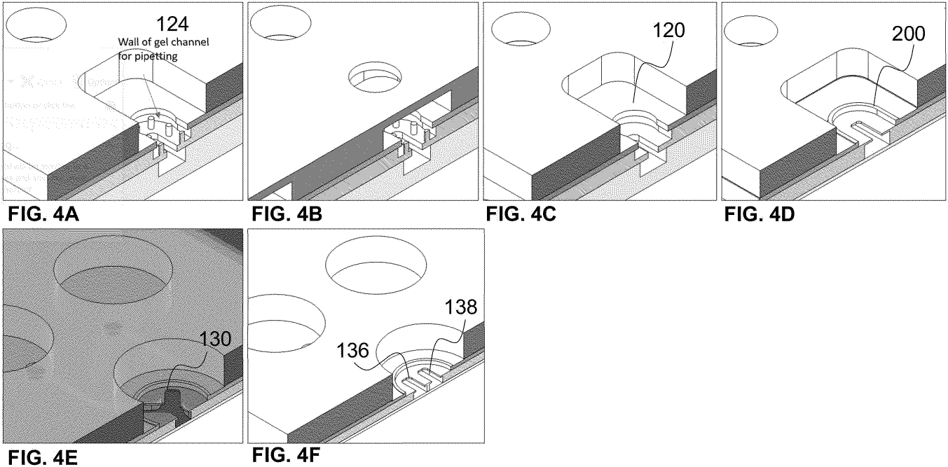

FIGS. 4A, 4B, 4C, 4D and 4E illustrate various example device configurations, with a detailed isometric view showing a cutaway of the hydrogel chamber. FIG. 4A shows an example configuration with an open top, while FIG. 4B shows an example configuration with a closed ceiling. FIG. 4C shows an example open-top configuration in which the hydrogel chamber 120 is absent of microposts. FIG. 4D shows an example configuration for forming a thin hydrogel membrane, in which the base surface of the media reservoir includes a geometrical meniscus-pinning feature 200. FIG. 4E shows an alternative example configuration for forming a thin hydrogel membrane over an aperture 130 residing above branched fluidic channels (as in FIG. 3B), while FIG. 4F shows an example configuration for forming a thin hydrogel membrane involving exposure of a membrane to multiple underlying fluidic channels through multiple apertures 136 and 138 (as in FIG. 3C).

The hydrogel or hydrogel membrane that is formed in-situ within the integrated hydrogel-supporting fluidic device can be employed as a scaffold to mimic biologically active tissues or organs. For example, many of the examples provided within the present disclosure demonstrate applications in which an in-situ fabricated hydrogel is employed to simulate the ECM of the airway tissue, and a set of microanchors and/or lip structures are employed to maintain structural integrity of the hydrogel in the presence of airflow-induced pressure.

In some example embodiments, an integrated hydrogel-supporting fluidic device may be integrated with a fluid delivery system (e.g. airflow system) that permits controlled injection of fluid, optionally with particulate matter, for studies such as air pollution studies.

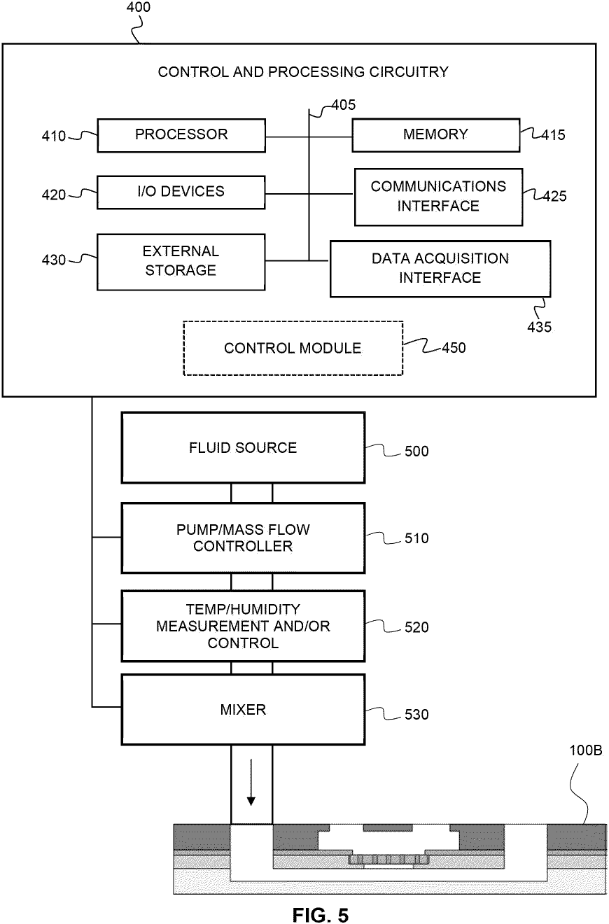

Referring now to FIG. 5, an example embodiment is illustrated in which an integrated hydrogel-supporting fluidic device 101B is integrated with a fluid delivery system. The example system includes control circuity 400 that is employed to control one or more fluidic delivery components. In the example implementation illustrated in FIG. 5, the system includes a fluid source 500, which may be a gas or a liquid, a pump or mass flow controller 510 that is connected to the fluid source 510 for the controlled delivery of fluid to the integrated hydrogel-supporting fluidic device 101B. The system may include one or more sensors and/or devices to modify the fluid prior to its delivery to the integrated hydrogel-supporting fluidic device 101B, such as, but not limited to, a temperature and/or humidity sensor and/or control device 520 (e.g. which may sense and/or modify one or more environmental properties of the fluid, such as temperature and/or humidity), and a mixer 530 for introducing an additional fluid or material into the fluid, such as, for example, particular matter. The mixer 530 may be in fluid communication with a source (not shown) of the additional fluid or material.

In some of the example embodiments described below, an airflow system is interfaced with the integrated hydrogel-supporting fluidic device, enabling flow rate control within the channel while delivering humidified air to maintain conditions that are favorable to airway epithelial function.[25] The example airflow systems described below also incorporates particulate delivery via physiological flow on the airway epithelium. The integration of the integrated hydrogel-supporting fluidic device with the airflow system allows accurate representation of the airway microenvironment and shows potential for many applications in respiratory research including air pollution and respiratory infection studies.

As shown in the figure, the example control and processing circuitry 400 may include a processor 410, a memory 415, a system bus 405, one or more input/output devices 420, and a plurality of optional additional devices such as communications interface 425, external storage 430, and a data acquisition interface 435. In one example implementation, a display (not shown) may be employed to provide a user interface for facilitating input to control the operation of the system 400. The display may be directly integrated into a control and processing device (for example, as an embedded display), or may be provided as an external device (for example, an external monitor).

The control and processing system 400 may include or be connectable to a console 480 that provides an interface for facilitating an operator to control one or more of the fluidic control devices, such as the pump/mass flow controller 510, and/or to monitor one or more sensor readings. The console may include, for example, one or more input devices, such, but not limited to, a keypad, mouse, joystick, touchscreen, and may optionally include a display device.

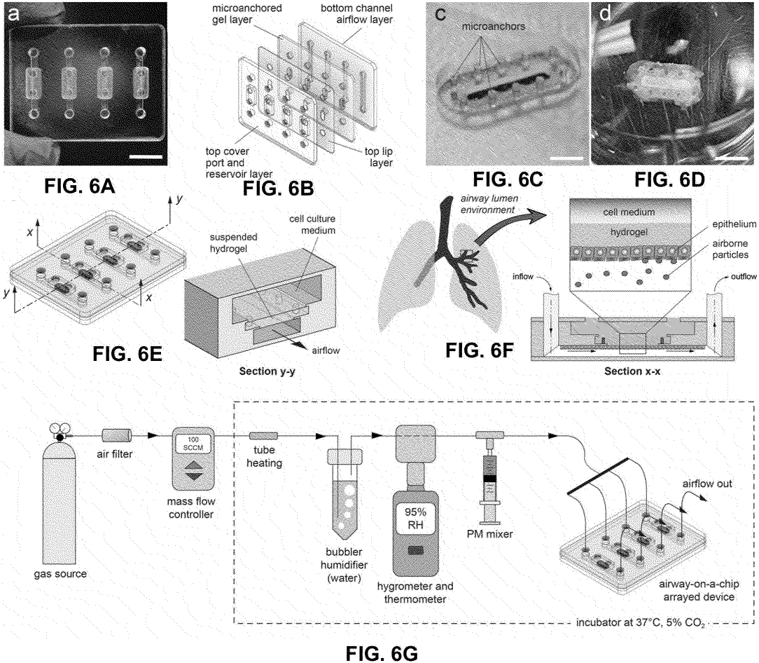

The methods described herein, such as methods for the controlled exposure of an in-situ-formed hydrogel to a test fluid, or other example methods described herein, can be implemented via processor 410 and/or memory 415. As shown in FIG. 6A, executable instructions represented as control module 450 are processed by control and processing circuitry 400. Such executable instructions may be stored, for example, in the memory 415 and/or other internal storage.

The methods described herein can be partially implemented via hardware logic in processor 410 and partially using the instructions stored in memory 415. Some embodiments may be implemented using processor 410 without additional instructions stored in memory 415. Some embodiments are implemented using the instructions stored in memory 415 for execution by one or more microprocessors. Thus, the disclosure is not limited to a specific configuration of hardware and/or software.

It is to be understood that the example system shown in the figure is not intended to be limited to the components that may be employed in a given implementation. For example, the system may include one or more additional processors. Furthermore, one or more components of control and processing circuitry 400 may be provided as an external component that is interfaced to a processing device. Furthermore, although the bus 405 is depicted as a single connection between all of the components, it will be appreciated that the bus 405 may represent one or more circuits, devices or communication channels which link two or more of the components. For example, the bus 405 may include a motherboard. The control and processing circuitry 400 may include many more or less components than those shown.

Some aspects of the present disclosure can be embodied, at least in part, in software, which, when executed on a computing system, transforms an otherwise generic computing system into a specialty-purpose computing system that is capable of performing the methods disclosed herein, or variations thereof. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version. Alternatively, the logic to perform the processes as discussed above could be implemented in additional computer and/or machine-readable media, such as discrete hardware components as large-scale integrated circuits (LSI’s), application-specific integrated circuits (ASIC’s), or firmware such as electrically erasable programmable read-only memory (EEPROM’s) and field-programmable gate arrays (FPGAs).

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

The multilayer integrated hydrogel-supporting fluidic devices of the present disclosure may be fabricated based on a wide variety of material platforms and methods. For example, device layers may be formed from materials such as, but not limited to, elastomers (e.g., PDMS), other thermoplastics (e.g., polystyrene, cyclo-olefin polymers (COPs) and cycle-olefin co-polymers (COCs), polytetrafluoroethylene (PTFE, or Teflon(™)), polycarbonate, acrylic or polymethylmethacrylate (PMMA)), and thermoplastic elastomers (“TPEs”). Surface treatment may be performed, depending on the material choice, in order to achieve a desired level of surface tension for supporting the hydrogel precursor solution over the aperture.

In example embodiments that employ anchoring protrusions, the contact angle between the hydrogel precursor solution and anchoring protrusion material should be considerably less than 90 degrees. The present inventors have found that surface treatment is unnecessary when PMMA is used as the device material, as PMMA is already mildly hydrophilic, and the protein content of typical hydrogel precursor solutions serves to further decrease the contact angle of the system. If similar geometry is to be fabricated from PDMS, surface treatment or oxygen plasma treatment may be almost employed to achieve reliable seeding of the hydrogel precursor solution.

While the present examples describe specific hydrogel precursor materials, it will be understood that a wide variety of hydrogel material systems may be employed to form an in-situ hydrogel. Non-limiting examples include biologically sourced gels that are commercially available such as Matrigel®, Cultrex® and Geltrex(™) derived from Engelbreth-Holm-Swarm (EHS) tumors, biologically sourced gels such as gelatin or Collagen I, either on its own or supplemented with other ECM components such as EHS tumor extract, purified laminin, other collagen types (IV, VII, etc.), or elastin, modified biomaterials to add features such as UV crosslinking such as gelatin methacryloyl (GeIMA), and synthetic or otherwise non-reactive hydrogels (polyethylene glycol (PEG), chitosan, alginate).

In some example implementations, one or more biomaterials may be incorporated into the gel composition to enhance the structural integrity of the gel, which may be beneficial, for example, to maintain gel integrity during extraction and handling of thinner gel constructs.

The present examples demonstrate the use of integrated hydrogel-supporting fluidic devices for airway tissue modelling, demonstrating arrayable and scalable devices that are amenable to withstand physiologic airflow. The examples show that device can be combined with a custom airflow system that permits controlled injection of particulate matter for air pollution studies. Results show that airflow is critical to efficiently achieving physiologic mimicry of airway epithelium composition, tight junction expression, mucus production, and cilia formation on epithelial cells. The examples below allow demonstrate how standard on-chip analysis while also permitting complete sample extraction and off-chip analysis via immunocytochemistry, microscopy, and histological sectioning and staining, thereby expanding the number and types of biological assays that can be employed.

Indeed, as demonstrated below, airflow on airway epithelium in the integrated hydrogel-supporting fluidic device was found to produce improved physiologic mimicry in airway epithelium composition, tight junction expression, mucus production, and cilia formation compared to submerged and static ALI cultures. Furthermore, the present examples show that integrated hydrogel-supporting fluidic device can be analyzed both on-chip to study particulate matter deposition as well as off-chip, after gel extraction, to enable immunocytochemistry, fluorescence and scanning electron microscopy, and histological sectioning and staining. The example integrated hydrogel-supporting fluidic devices and its extractability offers significant potential to study lung cell biology in new ways that can advance an understanding of particle-cell-matrix interactions and the effects of air pollution on lung disease.

In some example applications, airway epithelial cells may be exposed to different airflow rates, for longer airflow exposure times, or with different airflow directions (for mimicking breathing patterns) to examine how epithelial cell morphology and cell compositions are affected by various flow parameters. Second, airway smooth muscle cells may be embedded into the floating gel, or cultured on the top side of the gel (similar to previous work by the present inventors[27]), to shed light on epithelial-smooth muscle interactions that may be involved in the regulation of airway thickening and remodeling commonly associated with the onset of various CLDs such as asthma.[41-43] Third, particulate matter deposition onto epithelium and its effects on matrix remodeling and cell morphology may be studied by investigating deposition efficiency based on airflow rates and exposure times, and by analyzing the extracted gel using the various biological assays mentioned above. Such studies may have implications on the impact of air pollution on chronic lung disease development, and aid in the development of therapeutics to manage CLDs under adverse environmental conditions.

In some of the examples described below, an integrated hydrogel-supporting fluidic system was tested with only the Calu-3 cell line and a thick (~300 µm) floating gel, and further advances may include the use of stem-cell-derived or primary lung cells and the integration of thinner floating gels to better mimic the physiological cell and tissue microenvironments of native airways. Calu-3 cells were a convenient option to aid in the development of a cell culture protocol for the new integrated hydrogel-supporting fluidic system, while the thicker gel constructs helped to ensure gel integrity during sample handling.

In terms of mechanotransduction on airway epithelium, airway epithelial cells are constantly exposed to luminal shear stress caused by respiration. Shear stress, as well as other mechanical stimuli such as stretching and compression, is known to affect extracellular adenosine triphosphate (ATP) release, thereby regulating mucus secretion on the airway cilia.[41] In addition, studies have proposed various mechanisms of cilia response to mechano-stimulation, including via curvature-gated channels, strain-sensing molecules, stretch-sensitive channels connected to nearby microvilli, membrane tension-sensing molecules, shear stress-sensing membrane polymers, or internal shear-sensing molecules.[28] Similar phenomenon has been described previously in human umbilical vein endothelial cells (HUVECs), with surface-expressed glycocalyx providing mechanosensitivity to shear flow.[45] However, there remain open questions regarding the role of shear stress in airway epithelial cell differentiation and epithelial damage and repair mechanisms, thus providing an opportunity to apply the present example integrated hydrogel-supporting fluidic devices to explore these and other mechanistic questions.

In addition to the example applications described in the examples below, the present example integrated hydrogel-supporting fluidic devices and associated systems may be employed for a wide variety of applications and studies, including, but not limited to, lung airway modelling with airflow over epithelium and other lung cell types in coculture (e.g., endothelial cells, bronchial smooth muscle cells, mast cells), virus infection studies (e.g., COVID-19) for fundamental understanding of viral-particle-lung tissue interactions, cancer metastasis studies, including cell extravasation and intravasation through endothelial layers and underlying matrix (i.e., cell migration and cell invasion), cell invasion through basement membrane layer (cancer and immune cells), fundamental studies of molecular transport through ECM and through thin basement membranes, pre-clinical drug testing for diseases such as asthma and cancer (for anti-invasion therapy), construction of multilayered thin tissues structures (skin, intestinal wall, etc.) as model tissues for basic research complementing animal and human tissue models (i.e., all types of “organ-on-a-chip” systems), and usage of micro posts made of elastomeric materials such as PDMS to measure the contractile force from smooth muscle cells embedded in matrix.

The following examples are presented to enable those skilled in the art to understand and to practice embodiments of the present disclosure. They should not be considered as a limitation on the scope of the disclosure, but merely as being illustrative and representative thereof.

### EXAMPLES

### Example 1: Design and Fabrication of Example Integrated Hydrogel-Supporting Fluidic Device

The integrated hydrogel-supporting fluidic device described in the present non-limiting examples consists of four layers of poly(methylmethacrylate) (PMMA) plastic sheets that are first milled to create desired micro-geometric features and then bonded together by a liquid solvent bonding technique to create reliable bonds that remain leak-free throughout experimentation (FIG. 6A).[26] It will be understand that the present example multilayer configuration is but one example implementation of a fluid device configured for in-situ hydrogel formation, and that other device configurations may be employed, such as configurations that employ more or fewer layers.

In the present example device, the top layer contains the media reservoir located directly above the suspended gel, and also consists of the inlet and outlet access ports for loading hydrogel precursors, delivering cell culture media, and applying airflow to the microchannels (FIG. 6B).

The second layer, which was machined to a thickness of 300 µm using a facing operation, provides a protruding “lip” feature positioned above the upper gel surface. This lip feature is beneficial for preventing air leakage, a common occurrence with designs that do not include the lip whenever airflow is applied. Detachment of the gel is further prevented by providing additional surface area (beyond the side wall surfaces) for gel adhesion.

The third layer, which is faced to a thickness of 800 µm, contains the suspended hydrogel itself as well as anchoring microposts or “microanchors” that hold the hydrogel in position and withstand air pressure from the bottom channel while airflow is applied (FIG. 6C).

In the present experiments, the gel remained intact for > 48 h with flow rates of 0.6 cm³ s⁻¹, which based on the current design allowed the present inventors to essentially apply the full range of flow conditions found in the human lung inside the integrated hydrogel-supporting fluidic device.

Reinforcement of the hydrogel by microanchors is also advantageous because it secures the gel during culture and airflow but still allows the extraction of the gel for off-chip assessment after airflow exposure (FIG. 6D). Because of the microanchors, the integrated hydrogel-supporting fluidic device uses an extraction protocol can benefit from a more precise handling than the previous non-anchored design.

After gel extraction, the cell-coated gel construct can maintain its structural and mechanical integrity, allowing us to perform various sample manipulations including immunocytochemical staining, scanning electron microscopy (SEM), and histological sectioning to characterize the gel sample. Because of its location within the device, the suspended gel serves as an intermediary biological membrane that separates the upper media reservoir from the airway epithelial monolayer. The bottom side of the gel was chosen to be the culture site for the epithelium (FIGS. 6E, 6F) because the longer bottom microchannel was more advantageous for forming steady flow streamlines and avoiding disturbed flow patterns near the inlets and outlets during airflow.

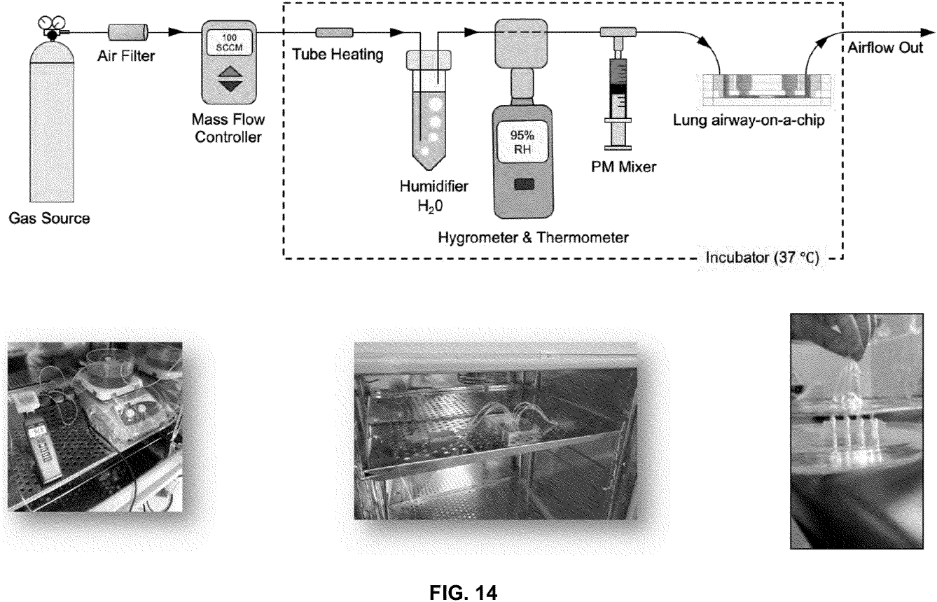

A schematic illustration and photographs of an example implementation of an extractable floating liquid-gel-based organ-on-a-chip system is provided in FIG. 14.

### Example 2: Interfacing Airflow System With Example Integrated Hydrogel-Supporting Fluidic Device

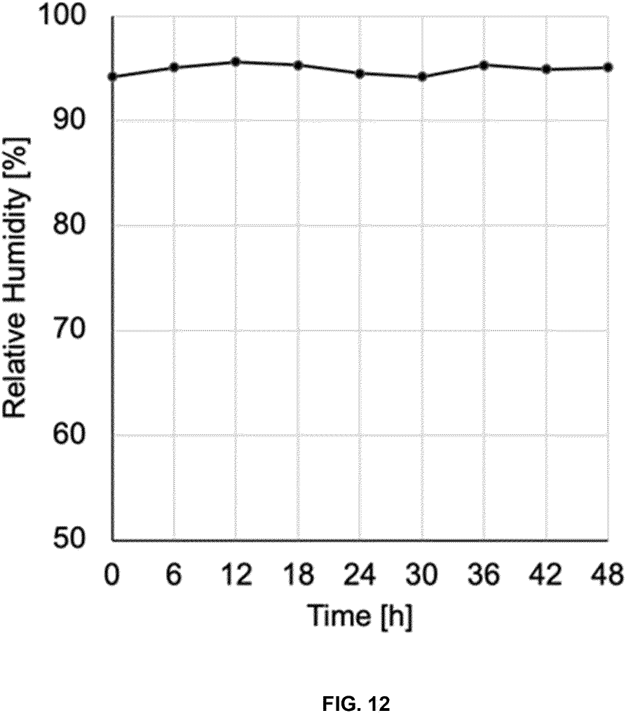

To provide airflow-mediated mechanotransduction on the airway epithelium in integrated hydrogel-supporting fluidic devices, an airflow system was custom-built with flow rate control, humidity control, inline particulate matter delivery, and humidity and temperature monitoring (FIG. 6G). The airflow source was a compressed gas cylinder of medical-grade air. The air was immediately filtered with an inline HEPA filter to minimize contamination. The outlet tubing from the mass flow controller (MFC) was heated in such a way as to ensure the air reaching the cells was 36.5 deg C or higher. The relative humidity of the air was maintained at 95 ± 2% for 48 h or more using a bubbler-type humidifier (FIG. 12). The entire airflow system was then placed inside a CO₂ incubator to prevent condensation inside the tubing.

### Example 3: Fabrication of Example Integrated Hydrogel-Supporting Fluidic Device

The example integrated hydrogel-supporting fluidic microdevice was fabricated from poly(methylmethacrylate) sheets (PMMA or acrylic, McMaster-Carr) that were micromachined and then solvent-bonded to create sealed devices. The toolpaths for the milling process were created using computer-aided design software Autodesk Fusion 360 (Autodesk Inc, CA, USA). The design file that contained the G-code for each layer was imported and used to direct the micromilling on an automated 3-axis computer numerical control (CNC) milling machine (P/N: PCNC770 Tormach, Waunakee, Wl, USA) that used different carbide endmills for different microchannels in the device.[46,47] The endmills used for fabrication of the example integrated hydrogel-supporting fluidic device were purchased from Caliber Industrial Supply (Mississauga, ON, Canada) and included the following: diameter 0.381 mm (P/N: 11101500, TuffCut- M.A. Ford), 3.175 mm (P/N: 211-214, MasterCut), 0.7938 mm (P/N: 209-202-1, MasterCut), 1.9844 mm (P/N: 30109, SOWA), and 1.5875 mm (P/N: 211-206, MasterCut). In total, the integrated hydrogel-supporting fluidic device was comprised of four layers of micromilled PMMA with various thicknesses (i.e., 1.5 mm, 0.3 mm, 0.8 mm, and 1.5 mm in order from top to bottom layer). Face operations were used to create the thin layers from original 1.5-mm stock sheets. The four PMMA layers were then bonded with a liquid solvent bonding technique.[48] 99% ethanol was carefully pipetted between the aligned PMMA layers, which were then placed between the platens of a heated hydraulic press (Carver Inc., Wabash, IN, USA). A compressive force of 1000 lbf over 4.9 cm² (i.e., the surface area of the device; equal to 200 psi pressure) at a temperature of 70° C. was applied for one minute.

### Example 4: Airflow System Setup

Compressed medical-grade air (P/N: 100034, Messer) was used as the air source. The source air flowed through a plastic in-line filter (P/N: 4795K42, McMaster-Carr) that removed airborne particles > 0.01 µm. The tubing was appropriately reduced in diameter to be fitted into the ports of the integrated hydrogel-supporting fluidic device (⅛” OD). The tubes used for the airflow system were purchased from McMaster-Carr: diameter ¼" ID / ⅜" OD, polyurethane rubber tubing (P/N: 5545K14), ⅛" ID / ¼" OD, PVC plastic tubing (P/N: 55485K72), ⅟16" ID / ⅛" OD, PVC plastic tubing (P/N: 5233K51).

After the air was filtered, the tube was connected to a mass flow controller (MFC) (Sierra 100 Smart-Trak, Sierra) to control the flow rate delivered to the device. The tube was locally heated to 70° C. to help increase the airstream temperature from room temperature to 37° C. A custom bubbler humidifier was placed inside the 37° C. incubator to produce saturated air with relative humidity of ~95%. Inside the bubbler humidifier, a porous stone was connected to the tube to produce microscale air bubbles. The tube that exited the humidifier was connected to a calibrated hygrometer (P/N: R6001, REED Instruments, Newmarket, ON, Canada) for real-time monitoring of the temperature and relative humidity. The sensor of the hygrometer was inserted into a custom-designed 3D-printed in-line adapter that was exposed to the oncoming airflow via a T-shaped junction. The tube that extended out of the hygrometer was connected to a 3D-printed manifold that divided the airflow into four smaller tubes that interfaced with the integrated hydrogel-supporting fluidic device.

For particulate matter delivery, a 3-way valve was set up in-line with the airflow system to connect the syringe containing carbon black (Vulcan XC-72R, FuelCell Store) in powder form. The carbon black was applied as a bolus injection into the air stream and then ultimately into the microfluidic channel. To confirm carbon black deposition, the cell-laden gel was extracted from the device and was fixed for scanning electron microscopy (SEM).

### Example 5: Flow Rate Setting

To select a representative flow rate, a 75-kg male exchanging 500 ml of tidal volume of air per breath was selected as a model. Dimensions for each of the airways were obtained from Filipovic et al and Weibel et al.[32] To mimic the shear stress on the epithelium in the device, wall shear stresses were calculated for all generations of the native airways, and then these shear stress values were used to back-calculate the average velocity required for the epithelium in the integrated hydrogel-supporting fluidic device to generate the same wall shear stress. Once integrated hydrogel-supporting fluidic device obtained a confluent epithelial monolayer, the airflow system was connected to the device and airflow of 0.0083 cm³ s⁻¹ was applied for either 24 h or 48 h.

A computational fluid dynamics (CFD) simulation was generated in COMSOL to check that the wall shear stress on the epithelium was uniform across the width of the gel. A simple conversion was used to calculate the flow velocity in the integrated hydrogel-supporting fluidic device bottom channels with given volumetric flow V_{avg} = Q/hw, where V_{avg} is average velocity in each channel, Q is the volumetric flow rate (m³ s⁻¹) given by the mass flow controller, h is the height of the channel (m), and w is the width of the channel (m).

### Example 6: Cell Culture

Calu-3 cells (ATCC® HTB-55TM) were cultured in MEM with Earle’s Salts (P/N: 320-026-CL, Wisent Bioproducts, Quebec, Canada) supplemented with 10% of fetal bovine serum (FBS, P/N: 26140079, Thermo Fisher Scientific, Waltham, MA, USA) and 1% of 10,000 U mL⁻¹ penicillin-streptomycin (P/N: 15140163, Thermo Fisher Scientific). Cell culture media was replenished every 48 hours and cells were subcultured when they reached ~70-80% confluent. The Calu-3 cells used in this study were subcultured up to ~15 passages.

### Example 7: Cell Culture in Example Integrated Hydrogel-Supporting Fluidic Device

The microchannels within the integrated hydrogel-supporting fluidic device were sterilized with 70% ethanol followed by washes of DPBS (-/-) (P/N: 14190144) and DPBS (+/+) (P/N: 14040133). After channels were dried, the gel pockets were coated with human plasma fibronectin (P/N: F0895-2 MG, Sigma Aldrich, St. Louis, MO, USA) with a concentration of 100 µg ml⁻¹ and incubated for 30 min at 37° C.

The floating hydrogel was prepared with a mixture of 6 mg ml⁻¹ Matrigel (phenol red-free, P/N: 356237, Corning), and 3 mg ml⁻¹ Type I collagen (rat-tail, P/N: CADB354249, VWR International, Radnor, PA, USA) at pH 7.4. The ratio of Matrigel to collagen was previously determined based on optimal cell viability.[27] Immediately after removing the fibronectin solution from the gel pocket, prepolymerized hydrogel solution (7 µl) was carefully pipetted into the gel pocket. The devices were incubated at 37° C. for 1 hour to polymerize the gel. After incubation, all the device channels were filled with cell culture media to rehydrate the gel for at least 1 day.

Calu-3 cells were trypsinized from the tissue culture flask and resuspended at 5 million cells ml⁻¹. 85 µl of cell suspension was pipetted into the bottom microchannel of the device and the device was then immediately flipped upside-down and placed on top of support blocks inside the incubator. After two hours of cell sedimentation and attachment, the cell culture media was replenished to remove unattached cells. Cell culture media was replenished every 24 hours until the end of the experiments.

After initial cell seeding, the suspended gel was closely monitored for cell confluency. Once the cells reached confluency on the gel, each device was tested under three different culture conditions: (i) submerged culture (epithelial cells submerged in liquid media) for 96 hours, (ii) static air-liquid interface (ALI) culture for 96 hours, and (iii) 48 hours of ALI culture followed by either 24 hours or 48 hours of airflow culture. ALI culture was achieved by removing the cell culture media from the bottom channel while maintaining media in the top reservoir channel.

### Example 8: Scanning Electron Microscopy

At the end of the culture experiment, the device was disassembled by cleaving each layer with a razor blade. The hydrogels were carefully extracted from the devices and were fixed in 4% paraformaldehyde (PFA) and 1% glutaraldehyde (GA) in 0.1 M phosphate buffer (pH 7.2) for at least 1 hour. The cells were then post-fixed with 1% Osmium Tetroxide and dehydrated using 50%, 60%, 70%, 90%, and 100% ethanol, consecutively. The samples were dried using critical point drying (CPD) machine (P/N: Autosamdri-810, Tousimis, Maryland, USA) and were coated with gold using Gold Sputter Coater (P/N: SC7640, Quorum Technologies, England). Images were taken at the Centre for Nanostructure Imaging (University of Toronto) using a scanning electron microscope (Quanta FEG 250 ESEM, FEI, Oregon) with various magnifications to observe the morphology of the epithelial surface. For the carbon black deposited SEM sample, energy dispersive spectroscopy (EDS) was performed using an EDAX silicon drift detector that scanned the region for carbon black particles.

### Example 9: Immunohistochemistry and Fluorescent Imaging

Cells were fixed with 4% PFA for 20 minutes at room temperature. Cells were permeabilized with 0.1% (v/v) Triton X for 3 minutes followed by blocking solution of 1% bovine albumin serum (BSA) for 30 minutes. Primary antibodies were diluted in the blocking solution and were applied to the cells overnight. Primary antibodies used included: MUC5AC monoclonal antibody (1:100, P/N: MA5-12178, Thermo Fisher Scientific), and ZO-1 polyclonal antibody (1:100, P/N: 40-2200, Thermo Fisher Scientific). The primary antibody was washed with PBS (+/+) 3 times in intervals of 10 minutes. Secondary antibodies (goat anti-rabbit IgG (Alexa Fluor 568, P/N: A11011, Thermo Fisher Scientific), goat antimouse IgG (Alexa Fluor 488, P/N: A11001, Thermo Fisher Scientific)) were applied for 30 minutes along with the Hoechst nuclear dye (1:1000, P/N: 33342, Thermo Fisher Scientific). Images were taken with Olympus^{®} IX-83 inverted microscope with ORCA® Flash 4.0 V2 camera. Images from each fluorescence channel were processed and merged using ImageJ software.

### Example 10: Histology Sectioning

Extracted gels were fixed in 4% PFA for 20 minutes and were submerged in 1X PBS. Samples were embedded in HistoGel™ and placed in 70% ethanol overnight. The samples were then processed in the tissue processor (Histo-Tek VP1, Sakura Finetek, USA). The samples were bisected and embedded using tissue embedder (Tissue-Tek®TECTM 6, Sakura Finetek, USA), which produced a paraffin block. Sections were cut with a thickness of 4 µm using a rotary microtome (P/N: HM 325, Epredia, USA) and were mounted on a glass slide. The sections on the slides were dried at 60∘C for 2 hours. Finally, sections were stained for hemotoxylin and eosin using standard protocols.

### Example 11: Statistical Analysis

For goblet cell differentiation, MUC5AC-expressing cells and total cells were manually counted, and the number of MUC5AC-expressing cells per 100 total cells was plotted. Goblet cell counts were obtained from three independent experiments (n = 3). One-way ANOVA with post hoc Tukey test was used to determine the statistical significance between the three culture conditions: (i) submerged, (ii) static ALI, and (iii) airflow.

### Example 12: Flow Rate Setting

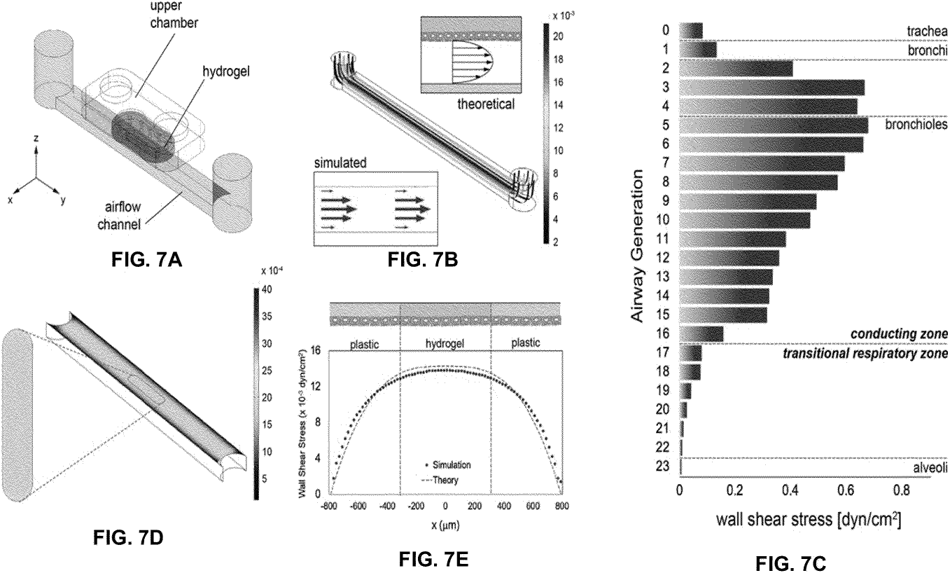

Deposition of particulate matter in lung airways depends on the size, density, and chemical composition of the particles. For instance, fine particulate matter with lower density tend to deposit deeper inside of the lungs compared with fine particulate matter with high density. Thus, versatility in the airflow system is necessary to model various situations involving particulate matter delivery, exposure, and deposition. Deng et al. modelled particle deposition and showed that particulate matter of diameter ~3 µm mostly deposited near the 20th airway generation (G20) of the respiratory tree.[28] Given that particulate matter of ~ 2.5 µm or smaller (referred to as PM2.5) can penetrate deep into the respiratory system and can exacerbate respiratory diseases such as asthma and lower respiratory inflammations,[29-31] the present inventors focused on airflow settings that matched the physiological flow conditions in lower respiratory airways (G19 to G22) (FIG. 7C) and the potential deposition of PM2.5.

A wall shear stress magnitude of 0.026 dyn cm⁻¹ was targeted on the epithelium, which based on a previous study by Weibel et al. mimics the shear stress in airway generation G20 of a 75-kg human with an estimated 1.0-L tidal volume.[32,33] To achieve 0.026 dyn cm⁻² in the integrated hydrogel-supporting fluidic device, the present inventors set the MFC to produce a volumetric airflow rate of 0.0083 cm³ s⁻¹ (or 0.5 cm³ min⁻¹). At this flow rate, the airflow Reynolds number was estimated to be Re ~ 0.2, resulting in laminar flow of air in the microchannel and a predictable parabolic velocity profile. With the 3D construction of the device (FIG. 7A) numerical simulations of the airflow in the bottom microchannel (using COMSOL) confirmed the parabolic velocity profile (FIG. 7B) and showed straight steady flow streamlines throughout the microchannel, indicating that the gel region was unaffected by any potential disturbed flow near the inlet and outlet ports. Furthermore, the gel region was exposed to uniform shear stress on its entire surface (FIG. 7D), with side wall effects only impacting epithelial cells that were adhered to the near-wall PMMA surfaces and not the gel surface (FIG. 7E).

### Example 13: Airflow Effects on Airway Epithelium

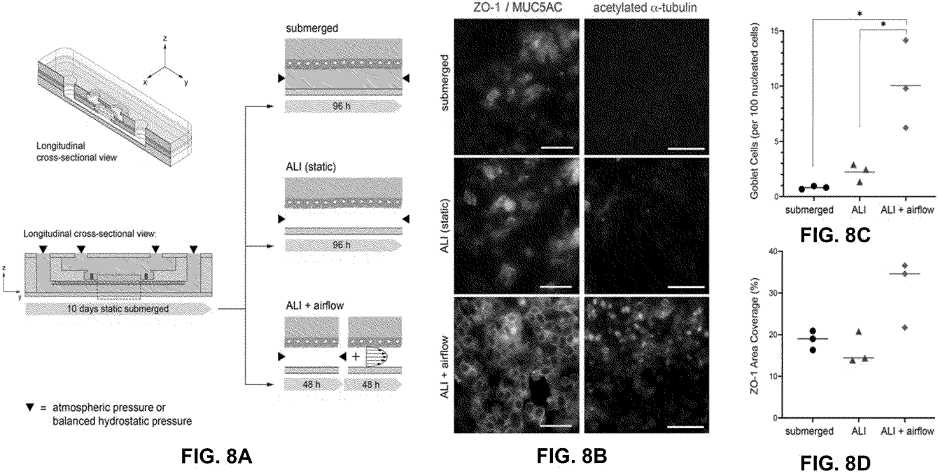

After assembling the airflow system and selecting the desired flow rate for mimicking shear in lower respiratory airways, the integrated hydrogel-supporting fluidic device was connected to the airflow system and tested the impact of airflow on morphology and function of the airway epithelium cultured on the floating gel. It was hypothesized that shear-induced mechanical stimulation caused by airflow over the airway epithelial cells (AECs) would offer a more physiologically relevant physical environment that would lead to improved epithelial monolayer formation, increased tight junction expression (and thus improved barrier function), and more representative airway epithelial cell composition, based on goblet cell population density. Three different culture conditions were compared on the gel-attached AECs: (i) submerged in cell culture media for 96 h (“submerged”); (ii) static air-liquid interface culture without airflow for 96 h (static ALI); and (iii) static ALI culture for 48 h followed immediately by airflow exposure with 0.026 dyn cm⁻² for an additional 48 h (ALI + airflow) (FIG. 8A).

AECs in submerged culture for 96 h showed diffuse cytoplasmic expression of ZO-1 with no localization on epithelial cell borders and did not display any acetylated α-tubulin expression (FIG. 8B). Similarly, AECs in static ALI culture for 96 h also did not exhibit any acetylated α-tubulin expression, while ZO-1 expression remained diffuse in the cytoplasm, with only slight localization near cell borders. In contrast, AECs cultured with airflow for 48 h (after static ALI culture of 48 h first) exhibited clear localization of ZO-1 tight junctions on epithelial cell borders, significant acetylated α-tubulin expression, as well as a significant increase in mucin-producing goblet cells (FIGS. 8C, 8D). Thus, only 48-h of airflow was sufficient to elicit strong epithelial cell response resulting in improved epithelial barrier structure, epithelial cell differentiation to goblet cells, and promotion of ciliated structures on epithelial cell surfaces. In particular, image analyses of cell type composition showed remarkable similarity between the airflow-stimulated sample and normal human lung airways in vivo, with the proportion of both goblet cells (∼10-15%, FIG. 8C) and ciliated epithelial cells (∼30%, see FIG. 8B, bottom right) matching closely with those of native airways.[34-36] In contrast, the fraction of mucin-producing goblet cells for the submerged (~1 in 100 cells) and static ALI (~2-3 in 100 cells) culture conditions were much lower and not representative of native lung cell compositions.

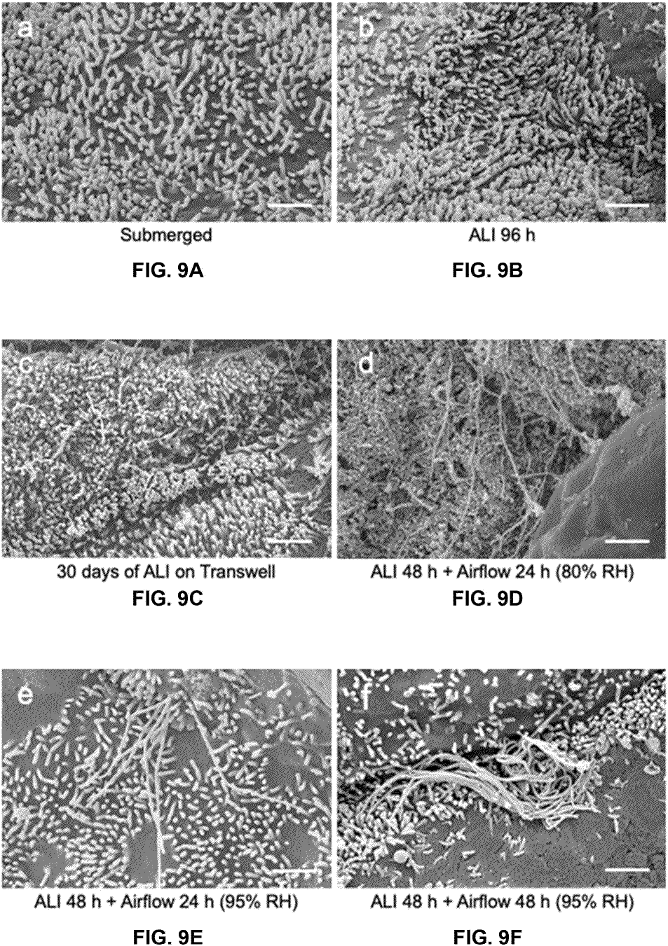

To characterize the cilia featured on the surface of AECs in the integrated hydrogel-supporting fluidic device, scanning electron microscopy (SEM) was performed of the cell-coated gel samples for all three culture conditions (FIGS. 9A-9F). On the integrated hydrogel-supporting fluidic device, AECs in submerged culture showed finger-like projections measuring ~0.5 µm in length, more commonly characterized as microvilli on the apical surface of epithelial cells (FIG. 9A). Airway cilia are expected to be ~5-7 µm long in the native respiratory tract but were not observed in the submerged condition. In several instances when culturing AECs in the submerged condition for >30 days, microvilli elongated to a range of ∼0.5-1 µm, but cilia were still not observed. Under static ALI conditions, AECs displayed very similar microvilli expression compared to the submerged condition (FIG. 9B). The present observations may be due to the shortened duration of ALI culture compared to conventional ALI culture of the Calu-3 cells reported in the literature, which is on the order of several weeks. Long-term ALI culture (30 days) on a Transwell insert also exhibited elongated microvilli ~1-µm in length, but longer cilia were again absent from the cell surface (FIG. 9C). Notably, AECs on the integrated hydrogel-supporting fluidic device cultured under static ALI conditions for 48 h followed by airflow for 24 h began exhibiting cilia that were ~5-6 µm long (FIG. 9E); and airflow for 48 h resulted in richer and longer cilia that were ~6-7 µm long (FIG. 9F). Thus, mechanostimulation by airflow led to clear differences in cilia formation and structure on the surface of epithelial cells. Motility and beating function of the cilia need to be further examined to verify the physiological accuracy more completely on this platform.

Viability of AECs cultured under all three conditions on the gel of the integrated hydrogel-supporting fluidic device remained high throughout the experiments. However, it was observed that the relative humidity of the air played a crucial role in both AEC viability and cilia expression. If relative humidity of the air dropped to 80%, the gel experienced surface dehydration leading to a significant reduction in cell viability to only ~20% after only 6-h of low-humidity airflow (FIG. 9D). This finding was in agreement with previous studies that showed the disruption of mucociliary function due to low relative humidity and temperature of the inspired air. [37]

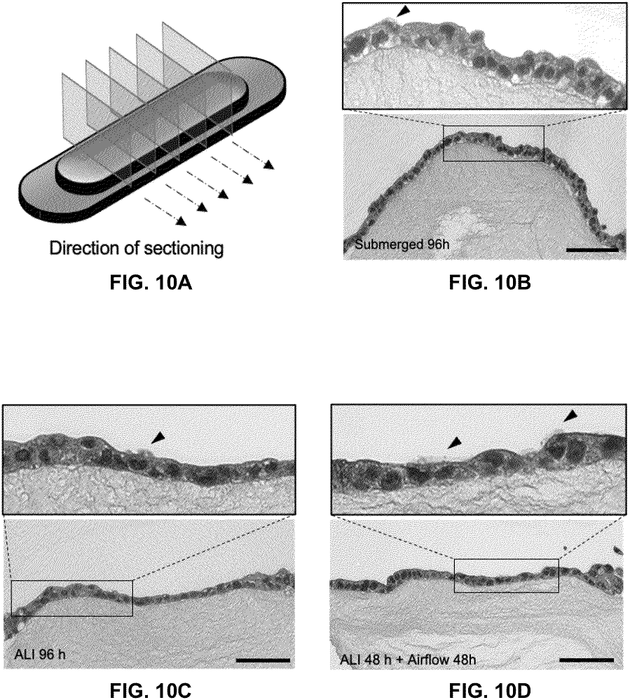

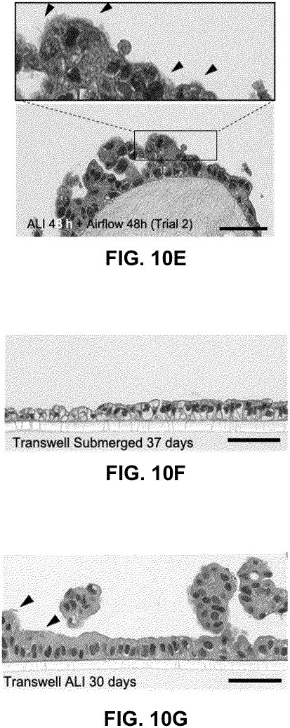

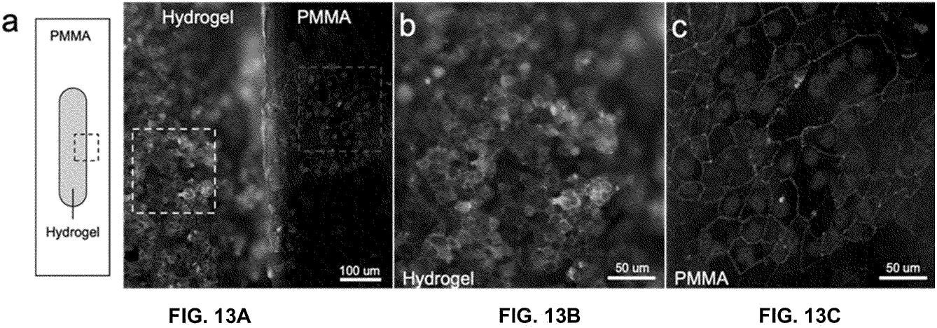

To demonstrate the utility of gel extraction for off-chip downstream analyses, the floating gel was extracted from the integrated hydrogel-supporting fluidic device for histology sectioning and hematoxylin and eosin (H&E) staining, with the goal of confirming epithelial morphology and examining the underlying matrix tissue structure under different experimental conditions (FIG. 10A). Based on cross-sections in the transverse direction (perpendicular to the direction of airflow), cells cultured under airflow for 48 h (two independent trials, FIGS. 10D-10E) showed more ciliated regions than cells cultured in the submerged or static ALI conditions (FIGS. 10B-10C). In addition, AECs in all three conditions showed cuboidal epithelial layer, which is different from the pseudostratified morphologies expected when airflow is applied. This may be evidence that pseudostratification occurs between 4 and 21 days, since others have reported pseudostratified epithelium after long-term culture (~21 days) under the ALI condition.[38,39] Interestingly, AECs in the integrated hydrogel-supporting fluidic device were cultured under airflow conditions that induced shear stress equivalent to that found in the lower airways (~G20) where epithelial cells are cuboidal and squamous.[40] While the effects of airflow on epithelial cell maturation and pseudostratification were not conclusive here, the histology sectioning experiments overall demonstrated the usefulness of implementing an extractable gel substrate for airway-on-a-chip systems that could reveal detailed tissue architecture consisting of epithelial cells lining the underlying matrix layer. This in vivo-like organization is significantly more physiologically relevant than cells cultured on the polymeric membranes of Transwell inserts, which can also be sectioned and stained, albeit without underlying tissue matrix (FIGS. 10F-10G). Notably, as can be observed in some samples, cells cultured on the biodegradable hydrogel appear to be multi-layered with nuclei closer together, while cells cultured on the polymeric surface were spread out to form thin and wide monolayers (FIGS. 13A-13C).

### Example 14: Particulate Matter Deposition

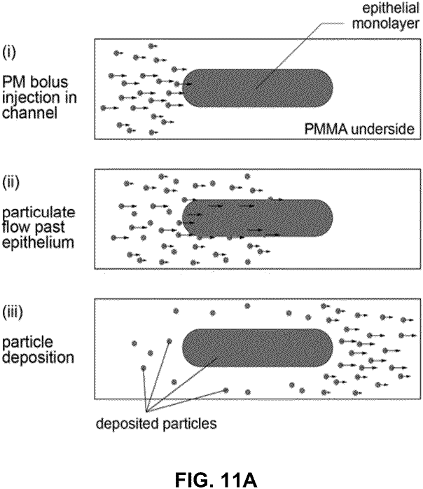

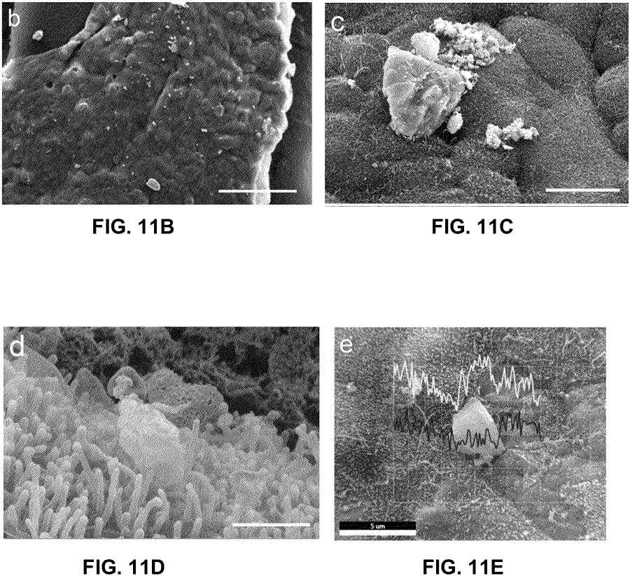

To demonstrate the potential of the platform to facilitate air pollution studies, particulate matter deposition was tested by delivering a dose of carbon black powder into the airstream for transport and deposition onto the airway epithelium in the integrated hydrogel-supporting fluidic device (FIG. 11A). After a single bolus injection of carbon black particles via airflow, the airflow was stopped and fixed the gel samples for off-chip image analysis. SEM images were taken of the epithelial surface to observe whether deposition of particles on the epithelium occurred (FIGS. 11B-11C). As shown, particles were deposited directly on the expressed airway microvilli and cilia (FIG. 11D). Moreover, an EDAX scan confirmed that the particles were indeed carbon black particles that were delivered, as evidenced by the peak level of carbon coincident with the location of the deposited particle (FIG. 11E).

### Example 15: Formation of Hydrogel Membrane

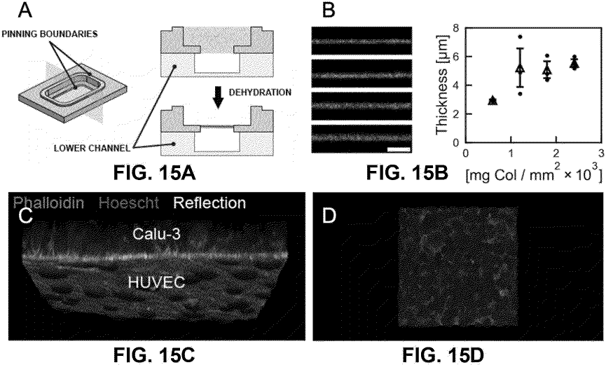

FIGS. 15A, 15B, 15C and 15D illustrate a fabrication methodology and preliminary characterization of collagen membrane devices. (A) The collagen membrane is fabricated in situ by first seeding a collagen hydrogel in the open-bottom well, situated directly above the lower channel, indicated in the left image and then allowing it to dry, producing a thin collagen film suspended in place. The hydrogel is maintained over the well opening by surface tension forces during polymerization. The membrane fabrication geometry can be bonded to any lower microchannel geometry prior to membrane seeding, an example of which is shown in light grey, thus enabling fluidic access to both the top surface through the open well and the bottom surface through the underlying channels. (B) Example optical cross-sections of collagen membranes, and the measured thickness variation as a function of the product of the initial hydrogel thickness and collagen concentration. (C, D) An example of a tissue model constructed using the device shown in A, consisting of adjacent monolayers of Calu-3 and human umbilical vein endothelial cells (HUVECs) separated by a collagen membrane.

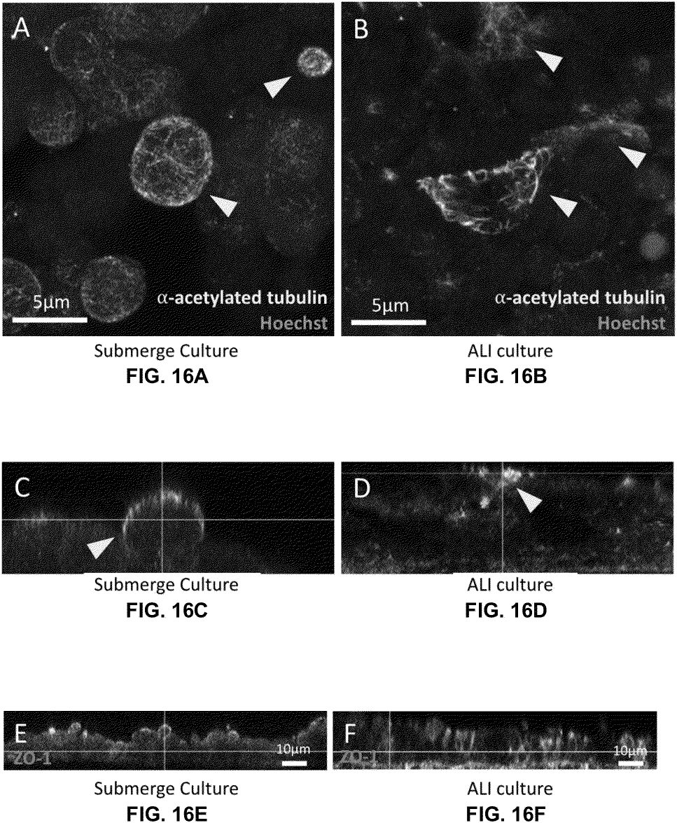

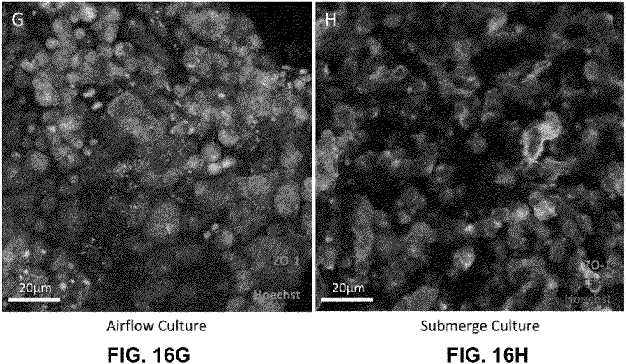

FIGS. 16A, 16B, 16C, 16D, 16E, 16F, 16G and 16H show morphologies of airway epithelial cells cultured under different conditions in the membrane-integrated E-FLOAT. (A, C, E) Images of submerge-cultured Calu-3 cells showing expressed ciliary structures and tight junctions. Notice that in C and E, which are cross-sectional images of Calu-3 cells, that the cells are cuboidal and not polarized, as ciliary structures (yellow = α-acetylated tubulin) are expressed around the cell membrane. Green = ZO-1 (tight junctions). (B, D, F) Images of the Calu-3 cells cultured under the air-liquid interface condition for 120 hours. Cross-sectional images show the ciliary structure that is expressed on the apical side of the cells as cells are polarized and pseudostratified. (G) Mucus-producing goblet cell differentiations (red = MUC5AC) under the airflow condition for 48 hours followed by 72 hours of ALI culture. (H) Mucus-producing goblet cell differentiations for submerge-cultured Calu-3 cells.

### Example 16: Bidirectional Air Flow System

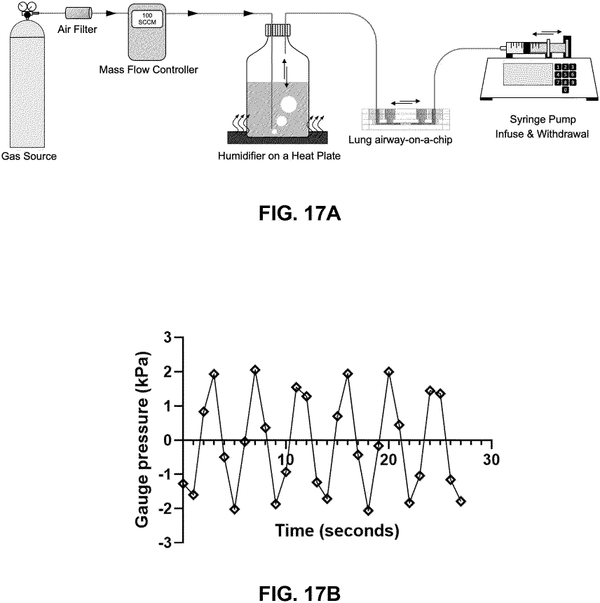

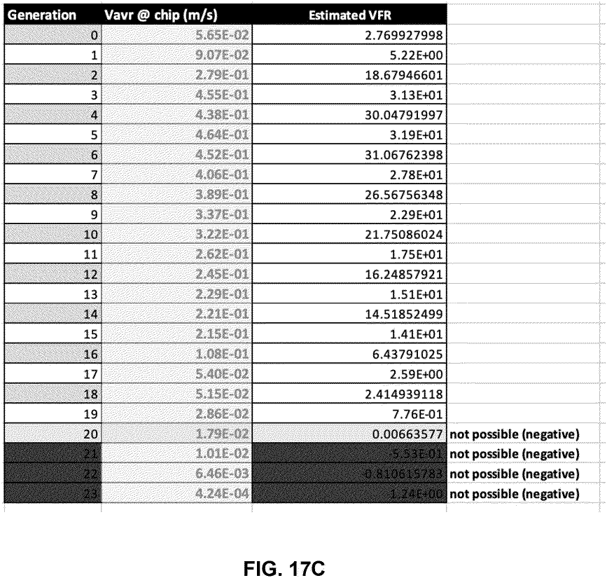

The airflow system described in the previous examples provides a unidirectional constant flow for a certain time. It can manipulate its flow rate but will always be flowing in one direction. However, to accurate mimic the breathing inside of the lung airways, it is essential to provide the bidirectional airflow on to the airway epithelial cells. The present example describes an adaptation of the airflow system to provide bidirectional functionality. The system includes the syringe pump, mass flow controller, humidity chamber and a gas cylinder, as shown in FIG. 17A. Using a pressure sensor, it was confirmed that positive and negative pressures were forming near the outlet of the microchannels. This confirms that there is a movement of air bidirectionally. The flow rate was measured at the same location and confirmed that the flow was measurable.

FIG. 17A provides a schematics of a bidirectional airflow system. The syringe pump is infusing and withdrawing the warm and humidified air to provide bidirectional airflow. The mass flow controller is humidifying the air contained in the humidifier.

FIG. 17B is a graph that shows the measured pressure near the outlet port of the device when 20 ml/min of air is infused/withdrew from the syringe pump.

FIG. 17C is a flow rate conversion chart. Lung has 23 generations of airways and each experience different flow rates. Vavr@chip(m/s) column shows the converted values of flow rate required in the device channel. The Estimated VFR column shows the volumetric flow rate that needs to be provided by the syringe pump in ml/min scale.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

### REFERENCES

WHO, Chron. Respir. Dis. 2007, 1.

S. Fereol, R. Fodil, G. Pelle, B. Louis, D. Isabey, Respir. Physiol. Neurobiol. 2008, 163, 3.

O. D. Chuquimia, D. H. Petursdottir, N. Periolo, C. Ferńndez, Infect. Immun. 2013, 81, 381.

M. E. Kreft, U. D. Jerman, E. Lasič, N. Hevir-Kene, T. L. Rižner, L. Peternel, K. Kristan, Eur. J. Pharm. Sci. 2015, 69, 1.

J. A. Dimasi, L. Feldman, A. Seckler, A. Wilson, Clin. Pharmacol. Ther. 2010, 87, 272.

T. G. O’Riordan, G. C. Smaldone, in Murray Nadel’s Textb. Respir. Med., 2015.

W. H. Chen, K. H. Lee, J. K. Mutuku, C. J. Hwang, Aerosol Air Qual. Res. 2018, 18, 866.

M. A. Mall, J. Aerosol Med. Pulm. Drug Deliv. 2008, 21, 13.

W. Stannard, C. O’Callaghan, J. Aerosol Med. Depos. Clear. Eff. Lung 2006, 19, 110.

M. B. Antunes, N. A. Cohen, Curr. Opin. Allergy Clin. Immunol. 2007, 7, 5.

V. K. Sidhaye, K. S. Schweitzer, M. J. Caterina, L. Shimoda, L. S. King, Proc. Natl. Acad. Sci. U. S. A. 2008, 105, 3345.

A. Pieterse, S. D. Hanekom, Multidiscip. Respir. Med. 2018, 13, 1.

A. A. Pezzulo, T. D. Starner, T. E. Scheetz, G. L. Traver, A. E. Tilley, B. G. Harvey, R. G. Crystal, P. B. McCray, J. Zabner, Am. J. Physiol. - Lung Cell. Mol. Physiol. 2011, 300, L25.

A. G. Buckley, K. Looi, T. losifidis, K. M. Ling, E. N. Sutanto, K. M. Martinovich, E. Kicic-Starcevich, L. W. Garratt, N. C. Shaw, F. J. Lannigan, A. N. Larcombe, G. Zosky, D. A. Knight, P. J. Rigby, A. Kicic, S. M. Stick, Biol. Proced. Online 2018, 20, 1.

D. Jiang, N. Schaefer, H. W. Chu, in Methods Mol. Biol., Humana Press Inc., 2018, 1809, 91-109.

G. Abraham, C. Zizzadoro, J. Kacza, C. Ellenberger, V. Abs, J. Franke, H. A. Schoon, J. Seeger, Y. Tesfaigzi, F. R. Ungemach, BMC Vet. Res. 2011, 7, 26.

N. E. T. Davidovich, Y. Kloog, M. Wolf, D. Elad, Biophys. J. 2011, 100, 2855.

D. Elad, U. Zaretsky, S. Avraham, R. Gotlieb, M. Wolf, I. Katra, S. Sarig, E. Zaady, Biomech. Model. Mechanobiol. 2018, 17, 891.

S. N. Bhatia, D. E. Ingber, Nat. Biotechnol. 2014, 32, 760.

E. W. Esch, A. Bahinski, D. Huh, Nat. Rev. Drug Discov. 2015, 14, 248.

S. Ahadian, R. Civitarese, D. Bannerman, M. H. Mohammadi, R. Lu, E. Wang, L. Davenport-Huyer, B. Lai, B. Zhang, Y. Zhao, S. Mandla, A. Korolj, M. Radisic, Adv. Healthc. Mater. 2018, 7, 2.

D. Huh, B. D. Matthews, A. Mammoto, M. Montoya-Zavala, H. Y. Hsin, D. E. Ingber, Science (80-.). 2010, 328, 1662.

P. Zamprogno, S. Wüthrich, S. Achenbach, G. Thoma, J. D. Stucki, N. Hobi, N. Schneider-Daum, C. M. Lehr, H. Huwer, T. Geiser, R. A. Schmid, O. T. Guenat, Commun. Biol. 2021, 4, 1, 12

A. O. Stucki, J. D. Stucki, S. R. R. Hall, M. Felder, Y. Mermoud, R. A. Schmid, T. Geiser, O. T. Guenat, Lab Chip 2015, 15, 1302.

E. Kilgour, N. Rankin, S. Ryan, R. Pack, Intensive Care Med. 2004, 30, 1491.

A. M. D. Wan, A. Sadri, E. W. K. Young, Lab Chip 2015, 15, 3785.

M. Humayun, C. W. Chow, E. W. K. Young, Lab Chip 2018, 18, 1298.

Q. Deng, L. Deng, Y. Miao, X. Guo, Y. Li, Environ. Res. 2019, 169, 237.

R. Habre, E. Moshier, W. Castro, A. Nath, A. Grunin, A. Rohr, J. Godbold, N. Schachter, M. Kattan, B. Coull, P. Koutrakis, J. Expo. Sci. Environ. Epidemiol. 2014, 24, 380.

A. Zanobetti, F. Dominici, Y. Wang, J. D. Schwartz, Environ. Heal. A Glob. Access Sci. Source 2014, 13, 1.

I. Kloog, B. Ridgway, P. Koutrakis, B. A. Coull, J. D. Schwartz, Epidemiology 2013, 24, 555.

E. R. Weibel, Am. J. Physiol. - Lung Cell. Mol. Physiol. 2013, 305, 6.

E. R. Weibel, B. Sapoval, M. Filoche, in Respir. Physiol. Neurobiol., Respir Physiol Neurobiol, 2005, 148, 1-2 SPEC. ISS, 3-21.

R. R. Mercer, M. L. Russell, V. L. Roggli, J. D. Crapo, Am. J. Respir. Cell Mol. Biol. 1994, 10, 613.

A. Wanner, M. Salathe, T. G. O’Riordan, Am. J. Respir. Crit. Care Med. 1996, 154, 1868.

K. H. Benam, R. Villenave, C. Lucchesi, A. Varone, C. Hubeau, H. H. Lee, S. E. Alves, M. Salmon, T. C. Ferrante, J. C. Weaver, A. Bahinski, G. A. Hamilton, D. E. Ingber, Nat. Methods 2016, 13, 2, 151-157.

J. E. C. Walker, R. E. Wells, Am. J. Med. 1961, 30, 259.

S. M. Jazaeri Farsani, M. Deijs, R. Dijkman, R. Molenkamp, R. E. Jeeninga, M. leven, H. Goossens, L. van der Hoek, Influenza Other Respi. Viruses 2015, 9, 51.

A. Dvorak, A. E. Tilley, R. Shaykhiev, R. Wang, R. G. Crystal, Am. J. Respir. Cell Mol. Biol. 2011, 44, 465.

R. G. Crystal, S. H. Randell, J. F. Engelhardt, J. Voynow, M. E. Sunday, in Proc. Am. Thorac. Soc., American Thoracic Society, 2008, 5, 7, 772-777.

B. Button, R. C. Boucher, Respir. Physiol. Neurobiol. 2008, 163, 189.

K. Yamamoto, T. Sokabe, N. Ohura, H. Nakatsuka, A. Kamiya, J. Ando, Am. J. Physiol. - Hear. Circ. Physiol. 2003, 285, 2 54-2.

D. C. Genetos, D. J. Geist, D. Liu, H. J. Donahue, R. L. Duncan, J. Bone Miner. Res. 2004, 20, 41.

M. J. Mondrinos, Y. S. Yi, N. K. Wu, X. Ding, D. Huh, Lab Chip 2017, 17, 3146.

Y. Zeng, X. F. Zhang, B. M. Fu, J. M. Tarbell, in Adv. Exp. Med. Biol., Springer New York LLC, 2018, 1097, 1-27.

D. Konstantinou, A. Shirazi, A. Sadri, E. W. K. Young, Sensors Actuators, B Chem. 2016, 234, 209.

D. J. Guckenberger, T. E. de Groot, A. M. D. Wan, D. J. Beebe, E. W. K. Young, Lab Chip 2015, 15, 2364.

A. M. D. Wan, T. A. Moore, E. W. K. Young, J. Vis. Exp. 2017, 2017, 55175.

Therefore what is claimed is:

1. A fluidic device comprising:
a multilayer fluidic structure having formed therein:
a fluidic channel;
a hydrogel chamber residing above said fluidic channel, said hydrogel chamber being defined at least in part by a side wall and a base surface, said base surface having an aperture defined therein such that said hydrogel chamber is in direct fluid communication with said fluidic channel through said aperture, and such that when a hydrogel is formed within said hydrogel chamber with the hydrogel contacting said base surface and extending across said aperture, said fluidic channel is in direct fluidic communication with a lower surface of the hydrogel in absence of an intervening membrane, the lower surface of the hydrogel being exposed to said fluidic channel through said aperture; and
a media reservoir residing above said hydrogel chamber, said media reservoir being in fluid communication with said hydrogel chamber, such that when the hydrogel is formed within said hydrogel chamber and liquid media is provided to said media reservoir, the liquid media is in fluid contact with an upper surface of the hydrogel;
said hydrogel chamber comprising a geometrical hydrogel retention structure configured to provide a restoring force to the hydrogel when fluidic pressure is applied to the lower surface of the hydrogel from said fluidic channel.

2. The fluidic device according to claim 1 wherein said hydrogel chamber and said aperture are configured such that when a hydrogel precursor solution is dispensed such that the hydrogel precursor solution contacts said base surface, the hydrogel precursor solution extends across said aperture without flowing into said fluidic channel, thereby facilitating in-situ formation of the hydrogel within said hydrogel chamber.

3. The fluidic device according to claim 1 wherein said geometrical hydrogel retention structure comprises a hydrogel retention lip extending from said side wall at a location remote from said base surface, such that when the hydrogel is formed within said hydrogel chamber with an upper surface of the hydrogel contacting a lower surface of the hydrogel retention lip, the hydrogel retention lip provides, at least in part, the restoring force to the hydrogel when fluidic pressure is applied to the lower surface of the hydrogel from said fluidic channel.

4. The fluidic device according to claim 1 wherein said geometrical hydrogel retention structure comprises one or more protrusions extending from said base surface, such that when the hydrogel is formed within the hydrogel chamber with the hydrogel at least partially surrounding the protrusions, the protrusions provide, at least in part, the restoring force to the hydrogel when fluidic pressure is applied to the lower surface of the hydrogel from said fluidic channel.

5. The fluidic device according to claim 1 further comprising the hydrogel within said hydrogel chamber.

6. The fluidic device according to claim 1 wherein said fluidic channel, said hydrogel chamber and said media reservoir define a first fluidic network, said multilayer fluidic structure comprising at least one additional fluidic network.

7. A method of forming a hydrogel in-situ within a fluidic device, the method comprising:
providing the fluidic device according to claim 1;
dispensing a hydrogel precursor solution such that the hydrogel precursor solution contacts said base surface and said geometrical hydrogel retention structure, and such that the hydrogel precursor solution extends across the aperture without flowing into the fluidic channel; and
hardening the hydrogel precursor solution to form the hydrogel in-situ within the hydrogel chamber, such that the hydrogel contacts, at least in part, the geometrical hydrogel retention structure.

8. The method according to claim 7 further comprising providing a cell-containing liquid to the fluidic channel and incubating the fluidic device to facilitate adhesion of cells of the cell-containing liquid to the lower surface of the hydrogel exposed by the aperture; and
providing liquid media to the media reservoir and incubating the fluidic device.

9. The method according to claim 8 further comprising:
removing the cell-containing liquid from said fluidic channel; and
delivering a fluid to the fluidic channel to expose the cells formed on the lower surface of the hydrogel to the fluid.

10. The method according to claim 9 wherein the fluid comprises a gas, and wherein the hydrogel is secured by the geometrical hydrogel retention structure such that a seal is maintained between the hydrogel and the fluidic channel during delivery of the gas.

11. The method according to claim 8 further comprising:
extracting the hydrogel from the hydrogel chamber, thereby obtaining an extracted hydrogel; and
performing one or more analytical procedures to characterize the cells of the extracted hydrogel.

12. A fluidic system comprising:
a fluidic device according to claim 1;
a fluid source;
a fluid delivery apparatus in fluid communication with said fluid source and an inlet of said fluidic channel; and
control circuitry operatively coupled to said fluid delivery apparatus, said control circuitry being configured to control said fluid delivery apparatus to deliver a fluid from said fluid source to said fluidic device.

13. A fluidic device comprising:
a multilayer fluidic structure having formed therein:
a fluidic channel;
a hydrogel chamber residing above said fluidic channel, said hydrogel chamber being defined at least in part by a side wall and a base surface, said base surface having an aperture defined therein such that said hydrogel chamber is in direct fluid communication with said fluidic channel through said aperture;
a media reservoir residing above said hydrogel chamber, said media reservoir being in fluid communication with said hydrogel chamber; and
a geometrical meniscus-pinning feature configured such that when a hydrogel precursor solution is delivered to said hydrogel chamber for in-situ formation of a hydrogel therein, said geometrical meniscus-pinning feature resists flow of the hydrogel precursor solution out of said hydrogel chamber, thereby preventing contact of the hydrogel precursor solution with one or more surfaces of said media reservoir;
said geometrical meniscus-pinning feature thereby confining formation of the hydrogel within said hydrogel chamber, such that subsequent drying of the hydrogel results in formation a hydrogel membrane secured to said base surface and extending over said aperture, and such that said fluidic channel is in direct fluidic communication with a lower surface of the hydrogel membrane in absence of an intervening additional membrane, the lower surface of the hydrogel membrane being exposed to said fluidic channel through said aperture.

14. The fluidic device according to claim 13 wherein said aperture is a first aperture and said base surface is a first base surface, and wherein said media reservoir is defined in part by a second base surface having a second aperture defined therein, such that said media reservoir is in fluid communication with said hydrogel chamber through said second aperture, and wherein said geometrical meniscus-pinning feature resides on a portion of said second base surface that lies adjacent to said second aperture.

15. The fluidic device according to claim 13 further comprising the hydrogel membrane extending over said aperture.

16. A method of forming a hydrogel membrane in-situ within a fluidic device, the method comprising:
providing the fluidic device according to claim 13;
dispensing a hydrogel precursor solution such that the hydrogel precursor solution contacts said base surface, and such that the hydrogel precursor solution extends across the aperture without flowing into the fluidic channel, and such that said geometrical meniscus-pinning feature prevents the hydrogel precursor solution from flowing into the media reservoir;
hardening the hydrogel precursor solution to form the hydrogel in-situ within the hydrogel chamber; and
drying, at least in part, the hydrogel to form the hydrogel membrane, wherein the geometrical meniscus-pinning feature facilitates shrinkage of the hydrogel within the hydrogel chamber to form the hydrogel membrane such that the hydrogel membrane is secured to the base surface and extends over the aperture.

17. The method according to claim 16 further comprising providing a cell-containing liquid to the fluidic channel and incubating the fluidic device to facilitate adhesion of cells of the cell-containing liquid to the lower surface of the hydrogel membrane exposed by the aperture;
providing liquid media to the media reservoir and incubating the fluidic device;
removing the cell-containing liquid from the fluidic channel; and
delivering a fluid to the fluidic channel to expose the cells formed on the lower surface of the hydrogel membrane to the fluid.

18. The method according to claim 17 wherein the fluid comprises a gas, and wherein the hydrogel membrane is secured by to the base surface of the hydrogel chamber such that a seal is maintained between the hydrogel membrane and the fluidic channel during delivery of the gas.

19. The method according to claim 18 further comprising:
extracting the hydrogel membrane from the hydrogel chamber, thereby obtaining an extracted hydrogel membrane; and
performing one or more analytical procedures to characterize the cells of the extracted hydrogel.

20. A fluidic system comprising:
a fluidic device according to claim 13;
a fluid source;
a fluid delivery apparatus in fluid communication with said fluid source and an inlet of said fluidic channel; and
control circuitry operatively coupled to said fluid delivery apparatus, said control circuitry being configured to control said fluid delivery apparatus to deliver a fluid from said fluid source to said fluidic device.
